SEQUENCE LISTING (6 sequences)

SEQ ID NO: 1 - DNA, 1448 bp, Pediococcus acidilactici
  misc_feature (6)..(6): n is a, c, g, or t
  misc_feature (24)..(24): n is a, c, g, or t
ttaganggctagctcctaaaaggntaccccaccggctttgggtgttacaaactctcatggtgtgacgggcggtgtgtacaaggcccgggaacgtattcaccgcggcatgctgatccgcgattactagcgattccgacttcgtgtaggcgagttgcagcctacagtccgaactgagaatggttttaagagattagctaaacctcgcggtttcgcgactcgttgtaccatccattgtagcacgtgtgtagcccaggtcataaggggcatgatgatttgacgtcgtccccaccttcctccggtttgtcaccggcagtctcactagagtgcccaactgaatgctggcaactagtaataagggttgcgctcgttgcgggacttaacccaacatctcacgacacgagctgacgacaaccatgcaccacctgtcattctgtccccgaagggaacgcctaatctcttaggttggcagaagatgtcaagacctggtaaggttcttcgcgtagcttcgaattaaaccacatgctccaccgcttgtgcgggcccccgtcaattcttttgagtttcaaccttgcggtcgtactccccaggcggattacttaatgcgttagctgcagcactgaagggcggaaaccctccaacacttagtaatcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtctcccagtttccaatgcacttcttcggttgagccgaaggctttcacattagacttaaaagaccgcctgcgctcgctttacgcccaataaatccggataacgcttgccacctacgtattaccgcggctgctggcacgtagttagccgtggctttctggttaaataccgtcactgggtgaacagttactctcacccacgttcttctttaacaacagagctttacgagccgaaacccttcttcactcacgcggcgttgctccatcagacttgcgtccattgtggaagattccctactgctgcctcccgtaggagtctgggccgtgtctcagtcccaatgtggccgattaccctctcaggtcggctacgcatcatcgccttggtgagccgttacctcaccaactagctaatgcgccgcgggtccatccagaagtgatagcagagccatcttttaaaagaaaaccaggcggttttctctgttatacggtattagcatctgtttccaggtgttatcccctgcttctgggcaggttacccacgtgttactcacccgtccgccactcacttcgtgttaaaatctcattcagtgcaagcacgtcataatcaattaacggaagttcgttcgactgca

SEQ ID NO: 2 - DNA, 1435 bp, Lactobacillus salivarius
  misc_feature (23)..(23): n is a, c, g, or t
tagacggctagctcctaaaaggntaccccaccggctttgggtgttacaaactctcatggtgtgacgggcggtgtgtacaaggcccgggaacgtattcaccgcggcatgctgatccgcgattactagcgattccgacttcgtgtaggcgagttgcagcctacagtccgaactgagaatggttttaagagattagctaaacctcgcggtttcgcgactcgttgtaccatccattgtagcacgtgtgtagcccaggtcataaggggcatgatgatttgacgtcgtccccaccttcctccggtttgtcaccggcagtctcactagagtgcccaactgaatgctggcaactagtaataagggttgcgctcgttgcgggacttaacccaacatctcacgacacgagctgacgacaaccatgcaccacctgtcattctgtccccgaagggaacgcctaatctcttaggttggcagaagatgtcaagacctggtaaggttcttcgcgtagcttcgaattaaaccacatgctccaccgcttgtgcgggcccccgtcaattcttttgagtttcaaccttgcggtcgtactccccaggcggattacttaatgcgttagctgcagcactgaagggcggaaaccctccaacacttagtaatcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtctcccagtttccaatgcacttcttcggttgagccgaaggctttcacattagacttaaaagaccgcctgcgctcgctttacgcccaataaatccggataacgcttgccacctacgtattaccgcggctgctggcacgtagttagccgtggctttctggttaaataccgtcactgggtgaacagttactctcacccacgttcttctttaacaacagagctttacgagccgaaacccttcttcactcacgcggcgttgctccatcagacttgcgtccattgtggaagattccctactgctgcctcccgtaggagtctgggccgtgtctcagtcccaatgtggccgattaccctctcaggtcggctacgcatcatcgccttggtgagccgttacctcaccaactagctaatgcgccgcgggtccatccagaagtgatagcagagccatcttttaaaagaaaaccaggcggttttctctgttatacggtattagcatctgtttccaggtgttatcccctgcttctgggcaggttacccacgtgttactcacccgtccgccactcacttcgtgttaaaatctcattcagtgcaagcacgtcataatcaattaacggaagtt

SEQ ID NO: 3 - DNA, 562 bp, Lactobacillus salivarius
  V_region (1)..(562)
tggagagtttgatcctggctcaggatgaacgctggcggcgtgcctaatacatgcaagtcgaacgaacttccgttaattgattatgacgtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttaaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggta

SEQ ID NO: 4 - DNA, 562 bp, Pediococcus pentosaceus; identical to SEQ ID NO: 3
  V_region (1)..(562)

SEQ ID NO: 5 - DNA, 562 bp, Lactobacillus salivarius; identical to SEQ ID NO: 3
  V_region (1)..(562)

SEQ ID NO: 6 - DNA, 562 bp, Lactobacillus salivarius; identical to SEQ ID NO: 3
  V_region (1)..(562)

(19) United States
(12) Patent Application Publication
(10) Pub. No.: US 2019/0075821 A1
(43) Pub. Date: Mar. 14, 2019

(54) Composition, Probiotic Formulation and Method to Reduce Bacterial Infection in Poultry

(71) Applicants: Novozymes A/S, Bagsvaerd (DK); The Board of Trustees of the University Arkansas, Little Rock, AR (US)

(72) Inventors: Ross Wolfenden, Fayetteville, AR (US); Billy Hargis, Fayetteville, AR (US); Amanda Wolfenden, Fayetteville, AR (US)

(73) Assignees: Novozymes A/S, Bagsvaerd (DK); The Board of Trustees of the University of Arkansas, Little Rock, AR (US)

(21) Appl. No.: 16/068,476

(22) PCT Filed: Jan. 25, 2017
(86) PCT No.: PCT/US2017/014838

§ 371 Date: Jul. 6, 2018

Related U.S. Application Data
(60) Provisional application No. 62/286,730, filed on Jan. 25, 2016.

(51) Int. Cl. A23K 10/18; A23K 50/75; A61K 35/747; A23L 33/135; A61P 31/04

(52) U.S. Cl. CPC A23K 10/18; A23K 50/75; A61P 31/04; A23L 33/135; A61K 35/747

(57) ABSTRACT

The present disclosure relates to compositions and probiotic formulations of bacterial isolates, and methods for selecting bacterial isolates capable of reducing levels of Salmonella and/or Campylobacter in poultry. Methods for reduction of Salmonella and/or Campylobacter in poultry are also part of the invention.

### REFERENCE TO A DEPOSIT OF BIOLOGICAL MATERIAL

This application contains a reference to a deposit of biological material, which deposit is incorporated herein by reference. For complete information see Example 1.

### REFERENCE TO SEQUENCE LISTING

This application contains a Sequence Listing in computer readable form. The computer readable form is incorporated herein by reference.

Index to sequence listing:
SEQ ID NO: 1 is 16S rDNA of NRRL B-50962 (Pediococcus acidilactici 64C1).
SEQ ID NO: 2 is 16S rDNA of NRRL B-67320 (Lactobacillus salivarius 64C2).
SEQ ID NO: 3 is 16S rDNA variable region of NRRL B-67178 (Lactobacillus salivarius 39B1).
SEQ ID NO: 4 is 16S rDNA variable region of NRRL B-67179 (Pediococcus pentosaceus 39B2).
SEQ ID NO: 5 is 16S rDNA variable region of NRRL B-67177 (Lactobacillus salivarius 59B).
SEQ ID NO: 6 is 16S rDNA variable region of NRRL B-67180 (Lactobacillus salivarius 34B).

### FIELD OF THE INVENTION

Compositions, probiotic formulations and methods are disclosed, including compositions and probiotic formulations of bacterial isolates, methods for selecting bacterial isolates capable of reducing levels of Salmonella and/or Campylobacter. Finally, methods for reduction of bacterial infections in poultry are disclosed.

### BACKGROUND OF THE INVENTION

Salmonella and Campylobacter are bacterial food borne pathogens common in poultry. These bacteria account for tens of thousands of cases of food borne illness in the United States annually. The USDA Food Safety and Inspection Service regulate poultry processing and inspect poultry processing to reduce the incidence of carcass contamination. Salmonella and/or Campylobacter contamination within a poultry processing plant are often the result of ante mortem infection of the poultry. During poultry processing the crop of the chicken is often ruptured. As the crop is often contaminated with both Salmonella and Campylobacter, it is the rupture of the crop that is the primary point from which poultry carcasses are contaminated.

Ante mortem intervention strategies exist that attempt to reduce or eliminate Salmonella and/or Campylobacter from carcasses. Chemical intervention methods are primarily composed of organic acids or volatile fatty acid compositions. Organic acids are capable of reducing these pathogens from the crop of poultry, but they can cause water refusal in the poultry. To reduce Salmonella and Campylobacter in the crop, the organic acids are added at elevated concentrations, typically in a concentration of approximately 0.5%. This concentration causes the flock to reduce, or in some cases cease, water consumption. Dehydration, and ultimately, a loss of carcass weight occurs. Animal welfare and economic concerns result. During this period a flock can lose in excess of two percent (2%) body weight. The result is a corresponding loss in carcass yield and a monetary loss to the producer.

Another group of products that can reduce Salmonella and Campylobacter from the lower GI tract of poultry includes long term probiotic regimes. Existing probiotic regimes are given several days or weeks prior to the slaughter date in order to reduce populations of these pathogens in the lower GI tract of poultry. These probiotic treatments also typically require multiple applications to work effectively.

The successful administration of chemical and probiotic treatment by growers and uptake by poultry is variable. The inconsistent administration is related to the several major shortcomings of current probiotics. For example, current probiotics require multiple applications during the life of a flock to reduce the presence of Salmonella at processing. Furthermore, required multiple applications of a chemical or probiotic product typically lead to greater cost of application.

Antibiotics have previously been used to reduce or eliminate Salmonella from the gastrointestinal tract of poultry prior to harvest. Antibiotics have withdrawal windows and cannot be used for a period ranging from a few days to a few weeks. Regulatory and social concerns have made the use of antibiotics less prevalent in recent years.

EP 1031287 discloses a poultry drinking water additive comprising monosaccharides and live bacteria of the genus Bacillus and a method for reducing harmful bacteria in the intestine of a layer. In U.S. Pat. No. 6,627,188 a cocktail containing water and a carbohydrate is disclosed for reducing bacterial enteropathogens in fowl.

Compositions and probiotic formulations for reducing Salmonella and/or Campylobacter in animals would be advantageous.

### SUMMARY OF THE INVENTION

The invention relates to a method for reducing the level of Salmonella and/or Campylobacter in poultry meat comprising the step of administering at least one bacterial isolate to the poultry less than 24 hours prior to slaughter such as, e.g., at the time of feed withdrawal or prior to the feed withdrawal period. The at least one bacterial isolate is preferably selected to reduce Salmonella and/or Campylobacter in the crop of poultry. In one embodiment the at least one bacterial isolate is from the genus Pediococcus, the genus Lactobacillus or a combination thereof. In a preferred embodiment the at least one bacterial isolate is from the species Pediococcus pentosaceus, the species Pediococcus acidilactici or the species Lactobacillus salivarius, or a combination thereof.

The invention further relates to bacterial isolates capable of reducing the level of Salmonella and/or Campylobacter in the crop of poultry such as Pediococcus pentosaceus, Pediococcus acidilactici or Lactobacillus salivarius. Compositions comprising the bacterial isolate according to the invention and an agriculturally acceptable excipient are also disclosed.

An additional embodiment of the invention is a method for selecting at least one bacterial isolate for reducing Salmonella and/or Campylobacter levels in poultry such as in the crop of poultry comprising the steps of:

i) identifying a bacterial isolate capable of reducing Salmonella and/or Campylobacter levels in vitro;

ii) testing said bacterial isolate in vivo; and

iii) selecting a bacterial isolate capable of reducing Salmonella and/or Campylobacter levels in vivo.

### BRIEF DESCRIPTION OF DRAWINGS

FIG. 1 illustrates a flow diagram of a selection process.

FIG. 2 illustrates a flow diagram for a crop assay.

FIG. 3 illustrates evaluation of lactic acid bacteria and isolates on Salmonella reduction in vitro with a crop assay.

FIG. 4 illustrates evaluation of isolates on Campylobacter reduction at 42° C. at pH 6.2 for 6 hours in an in vitro crop assay.

FIG. 5 illustrates evaluation of isolates on Campylobacter reduction at 42° C. at pH 7.2 for 6 hours in an in vitro crop assay.

FIG. 6 illustrates evaluation of lactic acid bacteria and combinations of lactic acid bacteria isolates on poultry pathogen reduction in an in vitro overlay assay.

FIG. 7 illustrates evaluation of lactic acid bacteria isolates on Salmonella enteritidis recovery from the crop of broiler chickens after an 8 hour feed withdrawal period in percent Salmonella enteritidis recovery.

FIG. 8 illustrates evaluation of lactic acid bacteria isolates on Salmonella enteritidis recovery from the crop of broiler chickens after an 8 hour feed withdrawal period illustrates colony forming units per gram (cfu/g; log₁₀).

FIG. 9 illustrates evaluation of candidate lactic acid bacteria on reducing Salmonella enteritidis in the crop of 4 week broilers after a 12 hour feed withdrawal period illustrates colony forming units per gram (cfu/g; log₁₀).

FIG. 10 illustrates evaluation of lactic acid bacteria isolates on Salmonella enteritidis recovery from the crop of broiler chickens after an 8 hour feed withdrawal period in percent Salmonella enteritidis recovery.

FIG. 11 illustrates evaluation of lactic acid bacteria isolates on Campylobacter jejuni recovery from the crop of broiler chickens after an 12 hour feed withdrawal period illustrates colony forming units per gram (cfu/g; log₁₀).

FIG. 12 illustrates evaluation of lactic acid bacteria isolates on Salmonella enteritidis recovery from the crop of broiler chickens after an 12 hour feed withdrawal period illustrates colony forming units per gram (cfu/g; log₁₀).

FIG. 13 illustrates evaluation of lactic acid bacteria isolates on Campylobacter jejuni recovery from the crop of broiler chickens after an 12 hour feed withdrawal period illustrates colony forming units per gram (cfu/g; log₁₀).

FIG. 14 illustrates percent Salmonella recovery from the crop of broiler chickens from in vivo field trial.

### DEFINITIONS

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

The term “about” means a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

The term “consisting of” means including, and limited to, whatever follows the phrase “consisting of.” Thus, the phrase “consisting of” indicates that the listed elements are required or mandatory, and that no other elements may be present. The term “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “administering,” or “administer” include providing a probiotic formulation of the disclosure to an animal by oral administration. Oral administration may be via drinking water, oral gavage, aerosol spray or animal feed.

A “decreased” or “reduced” amount is typically a “statistically significant” amount, and may include a decrease that is 1, 2, 3, 4, 5, 6, 7, 8, 9, 10, 15, 20, 30, 40, 50, 100, 200, 250, 500, 1000 or more times less, including all integers and decimal points in between and above 1 (e.g., 1.1, 1.2, 1.3, 1.4, 1.5, 1.6, 1.7, 1.8, 1.9), the amount produced by an animal in the absence of a probiotic formulation, for example, water without a probiotic formulation (e.g., the “native” or “natural” levels of Salmonella and/or Campylobacter). A decrease may include a decrease that is at least one log (Log₁₀) or more less than the amount produced by an animal in the absence of a probiotic formulation, for example, water without a probiotic formulation (e.g., the “native” or “natural” levels of Salmonella and/or Campylobacter of an animal). A decreased or reduced amount may include a decrease that is at least about 5 fold, 10 fold, 15 fold, 20 fold, 25 fold or 30 fold less than the amount produced by an animal in the absence of a probiotic formulation, for example, water without a probiotic formulation (e.g., the “native” or “natural” levels of Salmonella and/or Campylobacter).

A “decrease” in a response may be “statistically significant” as compared to the response produced by an animal in the absence of a probiotic formulation, for example, water without a probiotic formulation (e.g., the “native” or “natural” levels of Salmonella and/or Campylobacter of an animal), and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

“Bacterial isolate” as used herein means a bacterial strain which has been isolated from a live microbial source. A bacterial isolate is thus an isolate of a live bacterial microorganism but not, e.g., an enzymatically hydrolyzed protein, beef extract, yeast extract or autolyzed yeast. Examples of bacterial isolates of the invention include but are not limited probiotics, ie. live microorganisms that are providing health benefits when consumed.

The term “isolated” refers to a material that is substantially or essentially free from components that normally accompany it in its native state. For example, an isolated bacterial isolate may refer to a bacterial isolate that has been purified or removed from naturally or non-naturally occurring components that are present in its naturally occurring environment.

The term “modulate” includes to “decrease” one or more quantifiable parameters or indications, optionally by a defined and/or statistically significant amount. By “decrease” or “decreasing,” “reduce” or “reducing,” refers generally to the ability of a probiotic formulation to produce a lesser physiological response in an animal relative to the response caused by a control formulation. Relevant physical responses will be apparent to persons skilled in the art, and may include one or more of a decrease in levels of Salmonella and/or Campylobacter in an animal. A “decreased” or “reduced” amount is typically a “statistically significant” amount, and may include a decrease that is 1, 2, 3, 4, 5, 6, 7, 8, 9, 10, 15, 20, 30, 40, 50 or more times less (e.g., 100, 500, 1000 times), including all integers and decimal points in between and above 1 (e.g., 1.1, 1.2, 1.3, 1.4, 1.5, 1.6, 1.7, 1.8, 1.9), the amount produced by an animal in the absence of a probiotic formulation (e.g., the “native” or “natural” levels of Salmonella and/or Campylobacter of an animal). A decrease may include a decrease that is at least one log (Log₁₀) or more less than the amount produced by an animal in the absence of a probiotic formulation, for example, water without a probiotic formulation (e.g., the “native” or “natural” levels of Salmonella and/or Campylobacter of an animal). A decreased or reduced amount may include a decrease that is at least about 5 fold, 10 fold, 15 fold, 20 fold, 25 fold or 30 fold less than the amount produced by an animal in the absence of a probiotic formulation, for example, water without a probiotic formulation (e.g., the “native” or “natural” levels of Salmonella and/or Campylobacter).

As used herein, the terms “quantifying,” “quantification” or other related words refer to determining the quantity, mass, or concentration in a unit volume, of a parameter or indication, for example, levels of Salmonella and/or Campylobacter of an animal.

As used herein, the term “poultry” refers to domesticated birds. Poultry includes wildfowl, waterfowl, and game birds. Examples of poultry include, but are not limited to, chicken, broilers, bantams, turkey, duck, geese, guinea fowl, peafowl, quail, dove, pigeon (squab), and pheasant.

As used herein the term “agriculturally acceptable excipient” is a natural or synthetic substance formulated alongside the active ingredient of a formulation included for the purpose of long-term stabilization, bulking up solid formulations that contain potent active ingredients, or to confer a therapeutic enhancement on the active ingredient in the final dosage form, such as facilitating absorption, reducing viscosity, increasing viscosity, or enhancing solubility. In this context excipients may include but is not limited to skim milk powder, inulin, dextrose, vegetable protein powder, yeast extract, gums, citric acid, sodium ascorbate, wheat bran, rice hulls, calcium carbonate, chlorine binding agents, sugars, proteins powders, corn starch, starches, oligosaccharides, nitrite, nitrate, buffering agents, acidifying agents, etc.

### DETAILED DESCRIPTION OF THE INVENTION

The following description is merely exemplary in nature and is not intended to limit the present invention, its applications, or its uses. It should be understood that throughout the drawings, corresponding reference numerals indicate like or corresponding parts and features. The description of specific examples indicated in various embodiments of the present invention are intended for purposes of illustration only and are not intended to limit the scope of the invention disclosed herein. Moreover, recitation of multiple embodiments having stated features is not intended to exclude other embodiments having additional features or other embodiments incorporating different combinations of the stated features.

Furthermore, the detailed description of various embodiments herein makes reference to the accompanying drawing FIGS, which show various embodiments by way of illustration. While the embodiments are described in sufficient detail to enable those skilled in the art to practice the invention, it should be understood that other embodiments may be realized and that logical and mechanical changes may be made without departing from the spirit and scope of the present invention. Thus, the detailed description herein is presented for purposes of illustration only and not of limitation. For example, steps or functions recited in descriptions, any method, system, or process, may be executed in any order and are not limited to the order presented. Moreover, any of the step or functions thereof may be outsourced to or performed by one or more third parties. Furthermore, any reference to a singular component may include plural embodiments, and any reference to more than one component may include a singular embodiment.

Methods for Reduction of Salmonella and/or Campylobacter in Poultry

The invention relates to a method for reducing the level of Salmonella and/or Campylobacter in poultry meat comprising the step of administering at least one bacterial isolate to the poultry less than 24 hours prior to slaughter of said poultry preferably at the time of feed withdrawal or during the feed withdrawal period. Preferably the at least one bacterial isolate is selected to reduce Salmonella and/or Campylobacter in the crop of poultry. In one embodiment the at least one bacterial isolate is from Lactic Acid Bacteria. In one embodiment the at least one bacterial isolate is from the genus Pediococcus the genus Lactobacillus or a combination thereof. In an even more preferred embodiment the at least one bacterial isolate is from the species Pediococcus pentosaceus, the species Pediococcus acidilactici or the species Lactobacillus salivarius, or a combination thereof.

In a specific embodiment of the method the at least one bacterial isolate is selected from the group consisting:

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.

In a specific embodiment of the method the at least one bacterial isolate is Pediococcus acidilactici 64-C1 strain having the deposit accession number NRRL B-50962. In a specific embodiment of the method the at least one bacterial isolate is Lactobacillus salivarius 64-C2 strain having the deposit accession number NRRL B-67320. In a specific embodiment of the method the at least one bacterial isolate is Lactobacillus salivarius 39B1 strain having the deposit accession number NRRL B-67178. In a specific embodiment of the method the at least one bacterial isolate is Pediococcus pentosaceus 39B2 strain having the deposit accession number NRRL B-67179. In a specific embodiment of the method the at least one bacterial isolate is Lactobacillus salivarius 59B strain having the deposit accession number NRRL B-67177. In a specific embodiment of the method the at least one bacterial isolate is Lactobacillus salivarius 34B strain having the deposit accession number NRRL B-67180.

In one aspect the Salmonella is Salmonella enteritidis, Salmonella typhimurium, Salmonella heidelberg, Salmonella kentucky, or any combination thereof. In another aspect the Campylobacter is Campylobacter jejuni, Campylobacter coli, Campylobacter lari, Campylobacter upsaliensis, or any combination thereof. In another aspect the Salmonella is Salmonella enteritidis, Salmonella typhimurium, Salmonella heidelberg, Salmonella kentucky, or any combination thereof and the Campylobacter is Campylobacter jejuni, Campylobacter coli, Campylobacter lari, Campylobacter upsaliensis, or any combination thereof.

In a preferred embodiment the method reduces the level of Salmonella in the crop of poultry. In another preferred embodiment the method reduces the level of Campylobacter in the crop of poultry. In one embodiment the method reduces the level of Salmonella and the level of Campylobacter in the crop of poultry.

The administration of the bacterial isolate is preferably oral administration such as via the drinking water. The administration can be a single administration of a specified duration, such as, e.g., one administration of the bacterial isolate lasting the duration of feed withdrawal, such as, e.g., 12-24 hours prior to slaughter, and no further administrations later time point(s). The administration is preferably performed from 5 to 15 hours prior to slaughter, such as from 6 to 14 hours prior to slaughter, such as from 6 to 10 hours prior to slaughter. In another embodiment the administration is performed 7 to 14 hours prior to slaughter, such as from 8 to 14 hours prior to slaughter, such as from 8 to 13 hours prior to slaughter or from 9 to 13 hours prior to slaughter, such as from 8 to 12 hours prior to slaughter, from 9 to 12 hours prior to slaughter, or from 10 to 12 hours prior to slaughter. In another embodiment the administration is performed less than 10 hours prior to feed withdrawal, such as less than 9 hours prior to feed withdrawal, such as less than 8 hours prior to feed withdrawal, such as less than 7 hours prior to feed withdrawal, such as less than 6 hours prior to feed withdrawal, such as less than 5 hours prior to feed withdrawal, such as less than 4 hours prior to feed withdrawal, such as less than 3 hours prior to feed withdrawal, such as less than 2 hours prior to feed withdrawal, such as less than 1 hour prior to feed withdrawal, such as less than 30 minutes prior to feed withdrawal, or such as at the time of feed withdrawal.

In one embodiment the poultry is chicken, broilers, bantams, turkey, duck, geese, guinea fowl, peafowl, quail, dove, pigeon (squab), or pheasant, preferably chicken, turkey or duck.

The invention can result in that Salmonella and/or Campylobacter levels in the poultry meat or in the poultry crop are reduced by at least one log. In embodiments, the administration reduces Salmonella and/or Campylobacter levels in the poultry meat or the crop of poultry by at least one log (Log₁₀) compared to the levels of Salmonella and/or Campylobacter in the absence of this administration. In embodiments, the method reduces Salmonella and/or Campylobacter levels in the poultry meat or the poultry crop by at least about 0.50, about 1.00, about 1.50, about 2.00, about 2.50 or about 3.00 Logo, including any and all integers and decimal points in between and above 0.50 (e.g., 0.6, 0.7, 0.8, 0.9). In embodiments, the method decreases Salmonella and/or Campylobacter levels by at least about 5 fold, 10 fold, 15 fold, 20 fold, 25 fold or 30 fold, including all values in between, less than the amount produced in the absence of the composition, for example, water without the bacterial isolate.

In one embodiment the invention relates to a method for reduction of the levels of Salmonella and/or Campylobacter in the crop of poultry comprising administering the composition according to the invention to the poultry.

In various aspects, the invention relates to a method for reduction of the levels of Salmonella and/or Campylobacter in a poultry comprising the step of administering at least one bacterial isolate and at least one bacteriophage to poultry.

In various aspects, the bacterial isolate may be a lactic acid bacteria (LAB). In embodiments, the lactic acid bacteria includes, but is not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, Weisella, or any combination thereof.

The invention further relates to use of the composition according to the invention for reduction of the levels of Salmonella and/or Campylobacter in poultry such as in poultry meat after slaughter or in the crop of poultry.

### Bacterial Isolates

The invention relates to bacterial isolates capable of reducing the levels of Salmonella and/or Campylobacter in poultry. Preferably the bacterial isolates are capable of reducing the levels of Salmonella and/or Campylobacter in the crop of poultry, e.g., by at least one log (Log₁₀). In embodiments, the bacterial isolate may reduce Salmonella and/or Campylobacter levels by at least about 0.50, about 1.00, about 1.50, about 2.00, about 2.50 or about 3.00 Log₁₀, including any and all integers and decimal points in between and above 0.50 (e.g., 0.6, 0.7, 0.8, 0.9). In embodiments, the bacterial isolate decreases Salmonella and/or Campylobacter levels by at least about 5 fold, 10 fold, 15 fold, 20 fold, 25 fold or 30 fold, including all values in between, less than the amount produced in the absence of the composition, for example, water without the bacterial isolate.

In a preferred embodiment the Salmonella is Salmonella enteritidis, Salmonella typhimurium, Salmonella heidelberg, Salmonella kentucky, or any combination thereof. In another preferred embodiment the Campylobacter is Campylobacter jejuni, Campylobacter coli, Campylobacter lari, Campylobacter upsaliensis, or any combination thereof. In yet another preferred embodiment the Salmonella enteritidis, Salmonella typhimurium, Salmonella heidelberg, Salmonella kentucky, or any combination thereof and the Campylobacter is Campylobacter jejuni, Campylobacter coli, Campylobacter lari, Campylobacter upsaliensis, or any combination thereof.

In a preferred embodiment the bacterial isolate reduces the level of Salmonella and/or Campylobacter in the crop of poultry, e.g., by at least one log (Log₁₀). Preferably the at least one bacterial isolate is from Lactic Acid Bacteria. In one embodiment the at least one bacterial isolate is from the genus Pediococcus the genus Lactobacillus or a combination thereof. Preferably the bacterial isolate is from the species Pediococcus pentosaceus, the species Pediococcus acidilactici or the species Lactobacillus salivarius.

In a specific embodiment the bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.

The bacterial isolate is in one embodiment Pediococcus acidilactici 64-C1 strain having the deposit accession number NRRL B-50962.

The bacterial isolate is in one embodiment Lactobacillus salivarius 64-C2 strain having the deposit accession number NRRL B-67320.

The bacterial isolate is in one embodiment Lactobacillus salivarius 39B1 strain having the deposit accession number NRRL B-67178.

The bacterial isolate is in one embodiment Pediococcus pentosaceus 39B2 strain having the deposit accession number NRRL B-67179.

The bacterial isolate is in one embodiment Lactobacillus salivarius 59B strain having the deposit accession number NRRL B-67177.

The bacterial isolate is in one embodiment Lactobacillus salivarius 34B strain having the deposit accession number NRRL B-67180.

In a preferred embodiment the invention relates to a bacterial isolate selected from the group consisting of Pediococcus acidilactici 64-C1 deposited as NRRL B-50962, Lactobacillus salivarius 64-C2 deposited as NRRL B-67320, Lactobacillus salivarius 39B1 deposited as NRRL B-67178, Pediococcus pentosaceus 39B2 deposited as NRRL B-67179, Lactobacillus salivarius 59B deposited as NRRL B-67177, and Lactobacillus salivarius 34B deposited as NRRL B-67180, or a combination thereof.

The bacterial isolates have been deposited with the Agricultural Research Service Culture Collection (NRRL), 1815 North University Street, Peoria, Ill. 61604, U.S.A., under the above-identified accession numbers.

In various embodiments, the bacterial isolate is capable of reducing Salmonella and/or Campylobacter levels present in the crop of poultry. In embodiments, the bacterial isolate can reduce Salmonella and/or Campylobacter levels present in the crop of poultry and can survive environmental conditions within the crop. In embodiments, the bacterial isolate can survive at low pH conditions. In embodiments, low pH may be about pH 4 to about pH 7. In embodiments, low pH may be about pH 4.0, 4.5, 5.0, 5.5, 6.0, 6.5 or 7.0, including any and all integers and decimal points in between and above 4.0 (e.g., 4.6, 4.7, 4.8, 4.9).

### Compositions

In various aspects, a composition includes at least one bacterial isolate capable of reducing the levels of Salmonella and/or Campylobacter in poultry, and an agriculturally acceptable excipient. In embodiments, a composition includes at least one bacterial isolate capable of reducing the levels of Salmonella and/or Campylobacter in poultry.

In various aspects, a bacterial isolate may be a lactic acid bacteria (LAB). In embodiments, the lactic acid bacteria includes, but is not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, Weisella, or any combination thereof.

In embodiments, the composition comprises at least one bacterial isolate selected from the group consisting of

(a) a Pediococcus acidilactici 64-C1 strain having the deposit accession number NRRL B-50962;

(b) a Lactobacillus salivarius 64-C2 strain having the deposit accession number NRRL B-67320;

(c) a Lactobacillus salivarius 39B1 strain having the deposit accession number NRRL B-67178;

(d) a Pediococcus pentosaceus 39B2 strain having the deposit accession number NRRL B-67179;

(e) a Lactobacillus salivarius 59B strain having the deposit accession number NRRL B-67177; and

(f) a Lactobacillus salivarius 34B strain having the deposit accession number NRRL B-67180.

or a combination thereof.

In another preferred embodiment the composition comprises at least one bacterial isolate selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.

In various embodiments, the composition includes a bacterial isolate capable of reducing Salmonella and/or Campylobacter levels. In embodiments, the bacterial isolate reduces Salmonella and/or Campylobacter levels by at least one log (Log₁₀) compared to the levels of Salmonella and/or Campylobacter in the absence of the composition. In embodiments, the bacterial isolate may reduce Salmonella and/or Campylobacter levels by at least about 0.50, about 1.00, about 1.50, about 2.00, about 2.50 or about 3.00 Logo, including any and all integers and decimal points in between and above 0.50 (e.g., 0.6, 0.7, 0.8, 0.9). In embodiments, the bacterial isolate decreases Salmonella and/or Campylobacter levels by at least about 5 fold, 10 fold, 15 fold, 20 fold, 25 fold or 30 fold, including all values in between, less than the amount produced in the absence of the composition, for example, water without the composition.

In various embodiments, the composition includes a bacterial isolate capable of reducing Salmonella and/or Campylobacter levels present in the crop of poultry. In embodiments, the bacterial isolate can reduce Salmonella and/or Campylobacter levels present in the crop of poultry and can survive environmental conditions within the crop. In embodiments, the bacterial isolate can survive at low pH conditions. In embodiments, low pH may be about pH 4 to about pH 7. In embodiments, low pH may be about pH 4.0, 4.5, 5.0, 5.5, 6.0, 6.5 or 7.0, including any and all integers and decimal points in between and above 4.0 (e.g., 4.6, 4.7, 4.8, 4.9).

In various aspects, the composition includes at least one bacteriophage capable of reducing the levels of Salmonella and/or Campylobacter in poultry.

In various embodiments, Salmonella is Salmonella enteritidis, Salmonella typhimurium, Salmonella heidelberg, Salmonella kentucky, or any combination thereof.

In various embodiments, Campylobacter is Campylobacter jejuni, Campylobacter coli, Campylobacter lari, Campylobacter upsaliensis, or any combination thereof.

In various aspects, a composition includes at least one anti-Salmonella bacterial isolate. In various aspects, a composition includes at least one anti-Campylobacter bacterial isolate. In various aspects, a composition includes at least one anti-Salmonella bacterial isolate and at least one anti-Campylobacter bacterial isolate.

In a specific embodiment the composition comprises at least two bacterial isolates (such as at least three, such as at least four, such as at least five, such as at least six) selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.

In another specific embodiment the composition comprises at least two bacterial isolates (such as at least three, such as at least four, such as at least five, such as at least six) selected from the group consisting of

(a) a Pediococcus acidilactici 64-C1 strain having the deposit accession number NRRL B-50962;

(b) a Lactobacillus salivarius 64-C2 strain having the deposit accession number NRRL B-67320;

(c) a Lactobacillus salivarius 39B1 strain having the deposit accession number NRRL B-67178;

(d) a Pediococcus pentosaceus 39B2 strain having the deposit accession number NRRL B-67179;

(e) a Lactobacillus salivarius 59B strain having the deposit accession number NRRL B-67177; and

(f) a Lactobacillus salivarius 34B strain having the deposit accession number NRRL B-67180.

In a particularly preferred embodiment the composition comprises at least

(a) a Pediococcus acidilactici 64-C1 strain having the deposit accession number NRRL B-50962 and

(b) a Lactobacillus salivarius 64-C2 strain having the deposit accession number NRRL B-67320.

In another particularly preferred embodiment the composition comprises at least

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof and

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof.

### Selection Methods

Various aspects relate to methods of selecting bacterial isolates for reducing Salmonella and/or Campylobacter levels in poultry such as chickens.

In various aspects, a selection method for identifying a bacterial isolate capable of reducing Salmonella and/or Campylobacter levels is provided. In embodiments, a bacterial isolate is screened using an in vitro method. In embodiments, a bacterial isolate may be identified using selective agar plating. In embodiments, a bacterial isolate may be combined with Salmonella or Campylobacter and plated onto an agar plate to identify growth of the Salmonella or Campylobacter in the presence of the bacterial isolate compared to the growth of Salmonella or Campylobacter in the absence of a bacterial isolate.

In embodiments, a bacterial isolate capable of reducing Salmonella and/or Campylobacter levels may be further screened using an in vivo method. In embodiments, a bacterial isolate may be administered to a chicken and the number of Salmonella or Campylobacter recovered from the chicken may be determined and compared to the number recovered from a chicken not administered a bacterial isolate.

In various embodiments, a bacterial isolate may be capable of surviving various conditions, for example, various environments within the gastrointestinal tract of an animal. A bacterial isolate that survives various conditions is a bacterial isolate that maintains activity against and is capable of reducing levels of Salmonella and/or Campylobacter under such conditions. In embodiments, a bacterial isolate may be capable of surviving various pH ranges. In some embodiments, a bacterial isolate survives a pH range of pH 4.5 to pH 8.0. In embodiments, a bacterial isolate is capable of reducing Salmonella and/or Campylobacter levels at a pH range of pH 4.5 to pH 8.4. In embodiments, a bacterial isolate survives a pH of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In various embodiments, a bacterial isolate may be administered to a chicken prior to or at the time of feed withdrawal. In embodiments, the feed withdrawal period may be about 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, the feed withdrawal period may be about 8 hours or about 12 hours.

In embodiments, a bacterial isolate may be capable of reducing Salmonella and/or Campylobacter in the gastrointestinal tract of an animal. In embodiments, a bacterial isolate may be capable of reducing Salmonella and/or Campylobacter in the crop of poultry. The crop is a part of the esophagus where the initial stages of digestion occur. In one embodiment the poultry is chicken, broilers, bantams, turkey, duck, geese, guinea fowl, peafowl, quail, dove, pigeon (squab), or pheasant, preferably chicken, turkey or duck.

In various embodiments, a bacterial isolate may reduce Salmonella and/or Campylobacter levels by at least one log (Log₁₀). In embodiments, a bacterial isolate may reduce Salmonella and/or Campylobacter levels by at least about 0.50, about 1.00, about 1.50, about 2.00, about 2.50 or about 3.00 Log₁₀, including any and all integers in between.

In various embodiments, a bacterial isolate may be selected as specific for a single Salmonella and/or Campylobacter species. In embodiments, a single Salmonella and/or Campylobacter species may have been identified at a particular poultry farm, poultry house or poultry company. In embodiments, a bacterial isolate may be selected to specifically reduce the pre-identified Salmonella and/or Campylobacter species.

### PREFERRED EMBODIMENTS

Preferred embodiments of the invention are disclosed in the set of items herein below.

1. A method for reducing the level of Salmonella and/or Campylobacter in poultry meat comprising the step of administering at least one bacterial isolate to the poultry less than 24 hours prior to slaughter preferably at the time of feed withdrawal or during the feed withdrawal period.
2. The method of item 1, wherein the at least one bacterial isolate is selected to reduce Salmonella and/or Campylobacter in the crop of poultry.
3. The method of item 1 or 2, wherein the at least one bacterial isolate is from the species Pediococcus pentosaceus or the species Lactobacillus salivarius, or a combination thereof.
4. The method of any of items 1-3, wherein the at least one bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
5. The method of any of items 1-3, wherein the at least one bacterial isolate is Pediococcus acidilactici 64-C1 strain having the deposit accession number NRRL B-50962.
6. The method of any of items 1-3, wherein the at least one bacterial isolate is Lactobacillus salivarius 64-C2 strain having the deposit accession number NRRL B-67320.
7. The method of any of items 1-3, wherein the at least one bacterial isolate is from the species Lactobacillus salivarius 39B1 strain having the deposit accession number NRRL B-67178.
8. The method of any of items 1-3, wherein the at least one bacterial isolate is Pediococcus pentosaceus 39B2 strain having the deposit accession number NRRL B-67179.
9. The method of any of items 1-3, wherein the at least one bacterial isolate is Lactobacillus salivarius 59B strain having the deposit accession number NRRL B-67177.
10. The method of any of items 1-3, wherein the at least one bacterial isolate is Lactobacillus salivarius 34B strain having the deposit accession number NRRL B-67180.
11. The method of any of items 1-10, wherein the Salmonella is Salmonella enteritidis, Salmonella typhimurium, Salmonella heidelberg, Salmonella kentucky, or any combination thereof.
12. The method of any of items 1-11, wherein the Campylobacter is Campylobacter jejuni, Campylobacter coli, Campylobacter lari, Campylobacter upsaliensis, or any combination thereof.
13. The method of any of items 1-12, wherein the method reduces the level of Salmonella in the crop of poultry.
14. The method of any of items 1-13, wherein the method reduces the level of Campylobacter in the crop of poultry.
15. The method of any of items 1-14, wherein the administration is via the drinking water.
16. The method of any of items 1-15, wherein the administration is performed from 5 to 15 hours prior to slaughter, such as from 6 to 14 hours prior to slaughter, such as from 6 to 10 hours prior to slaughter.
17. The method of any of items 1-16, wherein the administration is performed less than 10 hours prior to feed withdrawal, such as less than 9 hours prior to feed withdrawal, such as less than 8 hours prior to feed withdrawal, such as less than 7 hours prior to feed withdrawal, such as less than 6 hours prior to feed withdrawal, such as less than 5 hours prior to feed withdrawal, such as less than 4 hours prior to feed withdrawal, such as less than 3 hours prior to feed withdrawal, such as less than 2 hours prior to feed withdrawal, such as less than 1 hour prior to feed withdrawal, such as less than 30 minutes prior to feed withdrawal, or such as at the time of feed withdrawal.
18. The method of any of items 1-17, wherein the poultry is chicken, broilers, bantams, turkey, duck, geese, guinea fowl, peafowl, quail, dove, pigeon (squab), or pheasant, preferably chicken, turkey or duck.
19. The method of any of items 1-18, wherein said Salmonella and/or Campylobacter levels are reduced by at least one log compared to the levels of Salmonella and/or

Campylobacter in the absence of the bacterial isolate.

20. The method of any of items 1-19, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
21. The method of any of items 1-20, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
22. The method of any of items 1-21, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
23. The method of any of items 1-22, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
24. The method of any of items 1-23, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
25. The method of any of items 1-24, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
26. The method of any of items 1-25, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).
27. A bacterial isolate reducing the level of Salmonella and/or Campylobacter in the crop of poultry.
28. The bacterial isolate of item 27, wherein the bacterial isolate is from the species Pediococcus pentosaceus or a Lactobacillus salivarius.
29. The bacterial isolate of item 27 or 28, wherein the bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
30. The bacterial isolate of any of items 27-29, wherein the bacterial isolate is Pediococcus acidilactici 64-C1 strain having the deposit accession number NRRL B-50962.
31. The bacterial isolate of any of items 27-29, wherein the bacterial isolate is Lactobacillus salivarius 64-C2 strain having the deposit accession number NRRL B-67320.
32. The bacterial isolate of any of items 27-29, wherein the bacterial isolate is from the species Lactobacillus salivarius 39B1 strain having the deposit accession number NRRL B-67178.
33. The bacterial isolate of any of items 27-29, wherein the bacterial isolate is Pediococcus pentosaceus 39B2 strain having the deposit accession number NRRL B-67179.
34. The bacterial isolate of any of items 27-29, wherein the bacterial isolate is Lactobacillus salivarius 59B strain having the deposit accession number NRRL B-67177.
35. The bacterial isolate of any of items 27-32, wherein the bacterial isolate is Lactobacillus salivarius 34B strain having the deposit accession number NRRL B-67180.
36. The bacterial isolate of any of items 27-35, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
37. The bacterial isolate of any of items 27-36, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
38. The bacterial isolate of any of items 27-37, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
39. The bacterial isolate of any of items 27-38, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
40. The bacterial isolate of any of items 27-39, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
41. The bacterial isolate of any of items 27-40, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
42. The bacterial isolate of any of items 27-41, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).
43. A composition comprising a bacterial isolate of any of items 27-42 and an agriculturally acceptable excipient.
44. The composition of item 43 comprising at least two bacterial isolates (such as at least three, such as at least four, such as at least five, such as at least six) selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
45. The composition of item 43 comprising at least two bacterial isolates selected from the group consisting of

(a) a Pediococcus acidilactici 64-C1 strain having the deposit accession number NRRL B-50962;

(b) a Lactobacillus salivarius 64-C2 strain having the deposit accession number NRRL B-67320;

(c) a Lactobacillus salivarius 39B1 strain having the deposit accession number NRRL B-67178;

(d) a Pediococcus pentosaceus 39B2 strain having the deposit accession number NRRL B-67179;

(e) a Lactobacillus salivarius 59B strain having the deposit accession number NRRL B-67177; and

(f) a Lactobacillus salivarius 34B strain having the deposit accession number NRRL B-67180.

46. The composition of item 45 comprising at least three bacterial isolates.
47. The composition of item 45 comprising at least four bacterial isolates.
48. The composition of item 45 comprising at least five bacterial isolates.
49. The composition of item 45 comprising at least six bacterial isolates.
50. A method for selecting at least one bacterial isolate for reducing Salmonella and/or Campylobacter levels in poultry comprising the steps of:

i) identifying a bacterial isolate capable of reducing Salmonella and/or Campylobacter levels in vitro;

ii) testing said bacterial isolate in vivo; and

iii) selecting a bacterial isolate of reducing Salmonella and/or Campylobacter levels in vivo.

51. The method of item 50, wherein the at least one bacterial isolate is from the species Pediococcus pentosaceus, the species Pediococcus acidilactici or the species Lactobacillus salivarius, or a combination thereof.
52. The method of item 50 or 51, wherein the Salmonella is Salmonella enteritidis, Salmonella typhimurium, Salmonella heidelberg, Salmonella kentucky, or any combination thereof.
53. The method of any of items 50-52, wherein the Campylobacter is Campylobacter jejuni, Campylobacter coli, Campylobacter lari, Campylobacter upsaliensis, or any combination thereof.
54. The method of any of items 50-53, wherein said Salmonella and/or Campylobacter levels are reduced by at least one log.

### FURTHER PREFERRED EMBODIMENTS

Further preferred embodiments of the invention are disclosed in the set of items herein below.

1. A method for reducing the level of Salmonella and/or Campylobacter in poultry meat comprising the step of administering at least one bacterial isolate to the poultry less than 24 hours prior to slaughter preferably at the time of feed withdrawal or during the feed withdrawal period.
2. The method of item 1 for reducing the level of Salmonella and Campylobacter in poultry meat.
3. The method of item 1 or 2, wherein the at least one bacterial isolate is selected to reduce Salmonella and/or Campylobacter in the crop of poultry.
4. A method for reducing the level of Salmonella and/or Campylobacter in poultry meat comprising the step of administering at least one bacterial isolate to the poultry less than 24 hours prior to slaughter preferably at the time of feed withdrawal or during the feed withdrawal period, wherein the at least one bacterial isolate is selected to reduce Salmonella and/or Campylobacter in the crop of poultry.
5. The method of item 4 for reducing the level of Salmonella and Campylobacter in poultry meat.
6. The method of any of items 1-5, wherein the at least one bacterial isolate is from Lactic Acid Bacteria.
7. The method of any of items 1-6, wherein the at least one bacterial isolate is from the genus Pediococcus or the genus Lactobacillus or a combination thereof.
8. The method of any of items 1-7, wherein the at least one bacterial isolate is from the species Pediococcus pentosaceus, the species Pediococcus acidilactici or the species Lactobacillus salivarius, or a combination thereof.
9. The method of any of items 1-8, wherein the at least one bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
10. The method of any of items 1-8, wherein the at least one bacterial isolate is Pediococcus acidilactici 64-C1 strain having the deposit accession number NRRL B-50962.
11. The method of any of items 1-8, wherein the at least one bacterial isolate is Lactobacillus salivarius 64-C2 strain having the deposit accession number NRRL B-67320.
12. The method of any of items 1-8, wherein the at least one bacterial isolate is from the species Lactobacillus salivarius 39B1 strain having the deposit accession number NRRL B-67178.
13. The method of any of items 1-8, wherein the at least one bacterial isolate is Pediococcus pentosaceus 39B2 strain having the deposit accession number NRRL B-67179.
14. The method of any of items 1-8, wherein the at least one bacterial isolate is Lactobacillus salivarius 59B strain having the deposit accession number NRRL B-67177.
15. The method of any of items 1-8, wherein the at least one bacterial isolate is Lactobacillus salivarius 34B strain having the deposit accession number NRRL B-67180.
16. The method of any of items 1-15, wherein the Salmonella is Salmonella enteritidis, Salmonella typhimurium, Salmonella heidelberg, Salmonella kentucky, or any combination thereof.
17. The method of any of items 1-16, wherein the Campylobacter is Campylobacter jejuni, Campylobacter coli, Campylobacter lari, Campylobacter upsaliensis, or any combination thereof.
18. The method of any of items 1-17, wherein the method reduces the level of Salmonella in the crop of poultry.
19. The method of any of items 1-17, wherein the method reduces the level of Campylobacter in the crop of poultry.
20. The method of any of items 1-17, wherein the method reduces the level of

Salmonella and Campylobacter in the crop of poultry.

21. The method of any of items 1-20, wherein the administration is via the drinking water.
22. The method of any of items 1-21, wherein the administration is in the form of a single administration.
23. The method of any of items 1-21, wherein the at least one bacterial isolate is administered to the poultry one time only.
24. The method of any of items 1-23, wherein the administration is performed from 5 to 15 hours prior to slaughter, such as from 6 to 14 hours prior to slaughter, such as from 6 to 10 hours prior to slaughter.
25. The method of any of items 1-23, wherein the administration is performed from 7 to 14 hours prior to slaughter, such as from 8 to 14 hours prior to slaughter, such as from 8 to 13 hours prior to slaughter or from 9 to 13 hours prior to slaughter, such as from 8 to 12 hours prior to slaughter, from 9 to 12 hours prior to slaughter, or from 10 to 12 hours prior to slaughter.
26. The method of any of items 1-25, wherein the administration is performed less than 10 hours prior to feed withdrawal, such as less than 9 hours prior to feed withdrawal, such as less than 8 hours prior to feed withdrawal, such as less than 7 hours prior to feed withdrawal, such as less than 6 hours prior to feed withdrawal, such as less than 5 hours prior to feed withdrawal, such as less than 4 hours prior to feed withdrawal, such as less than 3 hours prior to feed withdrawal, such as less than 2 hours prior to feed withdrawal, such as less than 1 hour prior to feed withdrawal, such as less than 30 minutes prior to feed withdrawal, or such as at the time of feed withdrawal.
27. The method of any of items 1-26, wherein the poultry is chicken, broilers, bantams, turkey, duck, geese, guinea fowl, peafowl, quail, dove, pigeon (squab), or pheasant, preferably chicken, turkey or duck.
28. The method of any of items 1-27, wherein said Salmonella and/or Campylobacter levels are reduced by at least one log compared to the levels of Salmonella and/or Campylobacter in the absence of the bacterial isolate.
29. The method of any of items 1-28, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
30. The method of any of items 1-29, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
31. The method of any of items 1-29, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
32. The method of any of items 1-29, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
33. The method of any of items 1-29, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
34. The method of any of items 1-29, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
35. The method of any of items 1-29, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).
36. A bacterial isolate reducing the level of Salmonella and/or Campylobacter in the crop of poultry.
37. A bacterial isolate of item 36, which is reducing the level of Salmonella and Campylobacter in the crop of poultry.
38. The bacterial isolate of item 36 or 37, wherein the at least one bacterial isolate is from Lactic Acid Bacteria.
39. The bacterial isolate of item 36 or 37, wherein the at least one bacterial isolate is from the genus Pediococcus or the genus Lactobacillus or a combination thereof.
40. The bacterial isolate of item 36 or 37, wherein the bacterial isolate is from the species Pediococcus pentosaceus, the species Pediococcus acidilactici or the species Lactobacillus salivarius, or a combination thereof.
41. The bacterial isolate of any of items 36-40, wherein the bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
42. The bacterial isolate of any of items 36-41, wherein the bacterial isolate is Pediococcus acidilactici 64-C1 strain having the deposit accession number NRRL B-50962.
43. The bacterial isolate of any of items 36-41, wherein the bacterial isolate is Lactobacillus salivarius 64-C2 strain having the deposit accession number NRRL B-67320.
44. The bacterial isolate of any of items 36-41, wherein the bacterial isolate is from the species Lactobacillus salivarius 39B1 strain having the deposit accession number NRRL B-67178.
45. The bacterial isolate of any of items 36-41, wherein the bacterial isolate is Pediococcus pentosaceus 39B2 strain having the deposit accession number NRRL B-67179.
46. The bacterial isolate of any of items 36-41, wherein the bacterial isolate is Lactobacillus salivarius 59B strain having the deposit accession number NRRL B-67177.
47. The bacterial isolate of any of items 36-41, wherein the bacterial isolate is Lactobacillus salivarius 34B strain having the deposit accession number NRRL B-67180.
48. The bacterial isolate of any of items 36-41, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
49. The bacterial isolate of any of items 36-41 or 48, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
50. The bacterial isolate of any of items 36-41 or 48, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
51. The bacterial isolate of any of items 36-41 or 48, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
52. The bacterial isolate of any of items 36-41 or 48, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
53. The bacterial isolate of any of items 36-41 or 48, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
54. The bacterial isolate of any of items 36-41 or 48, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).
55. A composition comprising a bacterial isolate of any of items 36-54 and an agriculturally acceptable excipient.
56. A composition comprising a bacterial isolate of any of items 36-54 and an agriculturally acceptable excipient which is selected from the group consisting of: Dry skim milk powder, inulin, fructo-oligosaccharides, soy protein, pea protein, yeast extract, sodium ascorbate, vitamin C, citric acid, mannan-oligosaccharides, dipotassium phosphate, phosphate buffers, lactose, trehalose and casein.
57. The composition of item 55 or 56 which does not comprise a monosaccharide.
58. The composition of item 55 or 56 which does not comprise a disaccharide.
59. The composition of item 55 or 56 which does not comprise a monosaccharide or a disaccharide.
60. The composition of any of items 55-59 comprising at least two bacterial isolates (such as at least three, such as at least four, such as at least five, such as at least six) selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
61. The composition of any of items 55-59 comprising at least two bacterial isolates selected from the group consisting of

(a) a Pediococcus acidilactici 64-C1 strain having the deposit accession number NRRL B-50962;

(b) a Lactobacillus salivarius 64-C2 strain having the deposit accession number NRRL B-67320;

(c) a Lactobacillus salivarius 39B1 strain having the deposit accession number NRRL B-67178;

(d) a Pediococcus pentosaceus 39B2 strain having the deposit accession number NRRL B-67179;

(e) a Lactobacillus salivarius 59B strain having the deposit accession number

NRRL B-67177; and

(f) a Lactobacillus salivarius 34B strain having the deposit accession number NRRL B-67180.

62. The composition of any of items 55-59 comprising at least three bacterial isolates.
63. The composition of any of items 55-59 comprising at least four bacterial isolates.
64. The composition of any of items 55-59 comprising at least five bacterial isolates.
65. The composition of any of items 55-59 comprising at least six bacterial isolates.
66. A method for selecting at least one bacterial isolate for reducing Salmonella and/or Campylobacter levels in poultry comprising the steps of:

i) identifying a bacterial isolate capable of reducing Salmonella and/or Campylobacter levels in vitro;

ii) testing said bacterial isolate in vivo; and

iii) selecting a bacterial isolate of reducing Salmonella and/or Campylobacter levels in vivo.

67. The method of item 66, wherein the at least one bacterial isolate is from Lactic Acid

Bacteria.

68. The method of item 66, wherein the at least one bacterial isolate is from the genus Pediococcus or the genus Lactobacillus or a combination thereof.
69. The method of item 68, wherein the at least one bacterial isolate is from the species Pediococcus pentosaceus, the species Pediococcus acidilactici or the species Lactobacillus salivarius, or a combination thereof.
70. The method of any of items 66-69, wherein the Salmonella is Salmonella enteritidis, Salmonella typhimurium, Salmonella heidelberg, Salmonella kentucky, or any combination thereof.
71. The method of any of items 66-69, wherein the Campylobacter is Campylobacter jejuni, Campylobacter coli, Campylobacter lari, Campylobacter upsaliensis, or any combination thereof.
72. The method of any of items 66-71, wherein said Salmonella and/or Campylobacter levels are reduced by at least one log.
73. A bacterial isolate for use in the treatment of poultry for reducing the level of Salmonella and/or Campylobacter in poultry meat, wherein the at least one bacterial isolate is administered to the poultry less than 24 hours prior to slaughter preferably at the time of feed withdrawal or during the feed withdrawal period.
74. The bacterial isolate of item 73 for reducing the level of Salmonella and Campylobacter in poultry meat.
75. The bacterial isolate of item 73 or 74, wherein the treatment reduces Salmonella and/or Campylobacter in the crop of poultry.
76. A bacterial isolate for use in the treatment of poultry for reducing the level of Salmonella and/or Campylobacter in poultry meat comprising the step of administering at least one bacterial isolate to the poultry less than 24 hours prior to slaughter preferably at the time of feed withdrawal or during the feed withdrawal period, wherein the at least one bacterial isolate is selected to reduce Salmonella and/or Campylobacter in the crop of poultry.
77. The bacterial isolate of item 76 for reducing the level of Salmonella and Campylobacter in poultry meat.
78. The bacterial isolate of any of items 73-77, wherein the at least one bacterial isolate is from Lactic Acid Bacteria.
79. The bacterial isolate of any of items 73-77, wherein the at least one bacterial isolate is from the genus Pediococcus or the genus Lactobacillus or a combination thereof.
80. The bacterial isolate of any of items 73-77, wherein the at least one bacterial isolate is from the species Pediococcus pentosaceus, the species Pediococcus acidilactici or the species Lactobacillus salivarius, or a combination thereof.
81. The bacterial isolate of any of items 73-77, wherein the at least one bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
82. The bacterial isolate of any of items 73-81, wherein the at least one bacterial isolate is Pediococcus acidilactici 64-C1 strain having the deposit accession number NRRL B-50962.
83. The bacterial isolate of any of items 73-81, wherein the at least one bacterial isolate is Lactobacillus salivarius 64-C2 strain having the deposit accession number NRRL B-67320.
84. The bacterial isolate of any of items 73-81, wherein the at least one bacterial isolate is from the species Lactobacillus salivarius 39B1 strain having the deposit accession number NRRL B-67178.
85. The bacterial isolate of any of items 73-81, wherein the at least one bacterial isolate is Pediococcus pentosaceus 39B2 strain having the deposit accession number NRRL B-67179.
86. The bacterial isolate of any of items 73-81, wherein the at least one bacterial isolate is Lactobacillus salivarius 59B strain having the deposit accession number NRRL B-67177.
87. The bacterial isolate of any of items 73-81, wherein the at least one bacterial isolate is Lactobacillus salivarius 34B strain having the deposit accession number NRRL B-67180.
88. The bacterial isolate of any of items 73-87, wherein the Salmonella is Salmonella enteritidis, Salmonella typhimurium, Salmonella heidelberg, Salmonella kentucky, or any combination thereof.
89. The bacterial isolate of any of items 73-87, wherein the Campylobacter is Campylobacter jejuni, Campylobacter coli, Campylobacter lari, Campylobacter upsaliensis, or any combination thereof.
90. The bacterial isolate of any of items 73-88, wherein the bacterial isolate reduces the level of Salmonella in the crop of poultry.
91. The bacterial isolate of any of items 73-87 or 89, wherein the bacterial isolate reduces the level of Campylobacter in the crop of poultry.
92. The bacterial isolate of any of items 73-91, wherein the bacterial isolate reduces the level of Salmonella and Campylobacter in the crop of poultry.
93. The bacterial isolate of any of items 73-92, wherein the administration is via the drinking water.
94. The bacterial isolate of any of items 73-93, wherein the administration is in the form of a single administration.
95. The bacterial isolate of any of items 73-93, wherein the at least one bacterial isolate is administered to the poultry one time only.
96. The bacterial isolate of any of items 73-95, wherein the administration is performed from 5 to 15 hours prior to slaughter, such as from 6 to 14 hours prior to slaughter, such as from 6 to 10 hours prior to slaughter.
97. The bacterial isolate of any of items 73-95, wherein the administration is performed from 7 to 14 hours prior to slaughter, such as from 8 to 14 hours prior to slaughter, such as from 8 to 13 hours prior to slaughter or from 9 to 13 hours prior to slaughter, such as from 8 to 12 hours prior to slaughter, from 9 to 12 hours prior to slaughter, or from 10 to 12 hours prior to slaughter.
98. The bacterial isolate of any of items 73-97, wherein the administration is performed less than 10 hours prior to feed withdrawal, such as less than 9 hours prior to feed withdrawal, such as less than 8 hours prior to feed withdrawal, such as less than 7 hours prior to feed withdrawal, such as less than 6 hours prior to feed withdrawal, such as less than 5 hours prior to feed withdrawal, such as less than 4 hours prior to feed withdrawal, such as less than 3 hours prior to feed withdrawal, such as less than 2 hours prior to feed withdrawal, such as less than 1 hour prior to feed withdrawal, such as less than 30 minutes prior to feed withdrawal, or such as at the time of feed withdrawal.
99. The bacterial isolate of any of items 73-98, wherein the poultry is chicken, broilers, bantams, turkey, duck, geese, guinea fowl, peafowl, quail, dove, pigeon (squab), or pheasant, preferably chicken, turkey or duck.
100. The bacterial isolate of any of items 73-99, wherein said Salmonella and/or Campylobacter levels are reduced by at least one log compared to the levels of Salmonella and/or Campylobacter in the absence of the bacterial isolate.
101. The bacterial isolate of any of items 73-100, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
102. The bacterial isolate of any of items 73-101, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
103. The bacterial isolate of any of items 73-101, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
104. The bacterial isolate of any of items 73-101, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
105. The bacterial isolate of any of items 73-101, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
106. The bacterial isolate of any of items 73-101, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
107. The bacterial isolate of any of items 73-101, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).
108. Use of at least one bacterial isolate in the manufacture of a medicament for reducing the level of Salmonella and/or Campylobacter in poultry wherein the at least one bacterial isolate is administered to the poultry less than 24 hours prior to slaughter, preferably at the time of feed withdrawal or during the feed withdrawal period.
109. Use of at least one bacterial isolate for reducing the level of Salmonella and/or Campylobacter in poultry meat comprising the step of administering at least one bacterial isolate to the poultry less than 24 hours prior to slaughter preferably at the time of feed withdrawal or during the feed withdrawal period.
110. The use of item 108 or 109 for reducing the level of Salmonella and Campylobacter in poultry meat. 111. The use of any of items 108-110, wherein the at least one bacterial isolate is selected to reduce Salmonella and/or Campylobacter in the crop of poultry.
112. Use of at least one bacterial isolate for reducing the level of Salmonella and/or Campylobacter in poultry meat comprising the step of administering at least one bacterial isolate to the poultry less than 24 hours prior to slaughter preferably at the time of feed withdrawal or during the feed withdrawal period, wherein the at least one bacterial isolate is selected to reduce Salmonella and/or Campylobacter in the crop of poultry.
113. The use of item 112 for reducing the level of Salmonella and Campylobacter in poultry meat.
114. The use of any of items 108-113, wherein the at least one bacterial isolate is from Lactic Acid Bacteria.
115. The use of any of items 108-113, wherein the at least one bacterial isolate is from the genus Pediococcus or the genus Lactobacillus or a combination thereof.
116. The use of any of items 108-113, wherein the at least one bacterial isolate is from the species Pediococcus pentosaceus, the species Pediococcus acidilactici or the species

Lactobacillus salivarius, or a combination thereof.

117. The use of any of items 108-113, wherein the at least one bacterial isolate is selected from the group consisting of

(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;

(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;

(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;

(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;

(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and

(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;

or any combination thereof.
118. The use of any of items 108-117, wherein the at least one bacterial isolate is Pediococcus acidilactici 64-C1 strain having the deposit accession number NRRL B-50962.
119. The use of any of items 108-117, wherein the at least one bacterial isolate is Lactobacillus salivarius 64-C2 strain having the deposit accession number NRRL B-67320.
120. The use of any of items 108-117, wherein the at least one bacterial isolate is from the species Lactobacillus salivarius 39B1 strain having the deposit accession number NRRL B-67178.
121. The use of any of items 108-117, wherein the at least one bacterial isolate is Pediococcus pentosaceus 39B2 strain having the deposit accession number NRRL B-67179.
122. The use of any of items 108-117, wherein the at least one bacterial isolate is Lactobacillus salivarius 59B strain having the deposit accession number NRRL B-67177.
123. The use of any of items 108-117, wherein the at least one bacterial isolate is Lactobacillus salivarius 34B strain having the deposit accession number NRRL B-67180.
124. The use of any of items 108-123, wherein the Salmonella is Salmonella enteritidis, Salmonella typhimurium, Salmonella heidelberg, Salmonella kentucky, or any combination thereof.
125. The use of any of items 108-123, wherein the Campylobacter is Campylobacter jejuni, Campylobacter coli, Campylobacter lari, Campylobacter upsaliensis, or any combination thereof.
126. The use of any of items 108-124, wherein the use reduces the level of Salmonella in the crop of poultry.
127. The use of any of items 108-123 or 125, wherein the use reduces the level of Campylobacter in the crop of poultry.
128. The use of any of items 108-127, wherein the use reduces the level of Salmonella and Campylobacter in the crop of poultry.
129. The use of any of items 108-128, wherein the administration is via the drinking water.
130. The use of any of items 108-129, wherein the administration is in the form of a single administration.
131. The use of any of items 108-129, wherein the at least one bacterial isolate is administered to the poultry one time only.
132. The use of any of items 108-131, wherein the administration is performed from 5 to 15 hours prior to slaughter, such as from 6 to 14 hours prior to slaughter, such as from 6 to 10 hours prior to slaughter.
133. The use of any of items 108-131, wherein the administration is performed from 7 to 14 hours prior to slaughter, such as from 8 to 14 hours prior to slaughter, such as from 8 to 13 hours prior to slaughter or from 9 to 13 hours prior to slaughter, such as from 8 to 12 hours prior to slaughter, from 9 to 12 hours prior to slaughter, or from 10 to 12 hours prior to slaughter.
134. The use of any of items 108-133, wherein the administration is performed less than 10 hours prior to feed withdrawal, such as less than 9 hours prior to feed withdrawal, such as less than 8 hours prior to feed withdrawal, such as less than 7 hours prior to feed withdrawal, such as less than 6 hours prior to feed withdrawal, such as less than 5 hours prior to feed withdrawal, such as less than 4 hours prior to feed withdrawal, such as less than 3 hours prior to feed withdrawal, such as less than 2 hours prior to feed withdrawal, such as less than 1 hour prior to feed withdrawal, such as less than 30 minutes prior to feed withdrawal, or such as at the time of feed withdrawal.
135. The use of any of items 108-134, wherein the poultry is chicken, broilers, bantams, turkey, duck, geese, guinea fowl, peafowl, quail, dove, pigeon (squab), or pheasant, preferably chicken, turkey or duck.
136. The use of any of items 108-135, wherein said Salmonella and/or Campylobacter levels are reduced by at least one log compared to the levels of Salmonella and/or Campylobacter in the absence of the bacterial isolate.
137. The use of any of items 108-136, wherein the at least one bacterial isolate has a 16S rDNA sequence comprising SEQ ID NO: 1, SEQ ID NO: 2, SEQ ID NO: 3, SEQ ID NO: 4, SEQ ID NO: 5 and/or SEQ ID NO: 6 or comprising a 16S rDNA sequence having at least 98% sequence identity to SEQ ID NO: 1 (such as at least 99% or 99.5% sequence identity to SEQ ID NO: 1, SEQ ID NO: 2, SEQ ID NO: 3, SEQ ID NO: 4, SEQ ID NO: 5 and/or SEQ ID NO: 6).
138. The use of any of items 108-137, wherein the at least one bacterial isolate has a 16S rDNA sequence comprising SEQ ID NO: 1 or comprising a 16S rDNA sequence having at least 98% sequence identity to SEQ ID NO: 1 (such as at least 99% or 99.5% sequence identity to SEQ ID NO: 1).
139. The use of any of items 108-137, wherein the at least one bacterial isolate has a 16S rDNA sequence comprising SEQ ID NO: 2 or comprising a 16S rDNA sequence having at least 98% sequence identity to SEQ ID NO: 2 (such as at least 99% or 99.5% sequence identity to SEQ ID NO: 2).
140. The use of any of items 108-137, wherein the at least one bacterial isolate has a 16S rDNA sequence comprising SEQ ID NO: 3 or comprising a 16S rDNA sequence having at least 98% sequence identity to SEQ ID NO: 3 (such as at least 99% or 99.5% sequence identity to SEQ ID NO: 3).
141. The use of any of items 108-137, wherein the at least one bacterial isolate has a 16S rDNA sequence comprising SEQ ID NO: 4 or comprising a 16S rDNA sequence having at least 98% sequence identity to SEQ ID NO: 4 (such as at least 99% or 99.5% sequence identity to SEQ ID NO: 4).
142. The use of any of items 108-137, wherein the at least one bacterial isolate has a 16S rDNA sequence comprising SEQ ID NO: 5 or comprising a 16S rDNA sequence having at least 98% sequence identity to SEQ ID NO: 5 (such as at least 99% or 99.5% sequence identity to SEQ ID NO: 5).
143. The use of any of items 108-137, wherein the at least one bacterial isolate has a 16S rDNA sequence comprising SEQ ID NO: 6 or comprising a 16S rDNA sequence having at least 98% sequence identity to SEQ ID NO: 6 (such as at least 99% or 99.5% sequence identity to SEQ ID NO: 6).

### EXAMPLES

### Identification, Characterization and Deposit of the Biological Material

The following biological materials were deposited under the terms of the Budapest Treaty at Agricultural Research Service Culture Collection (NRRL), 1815 North University Street, Peoria, Ill. 61604, U.S.A., and given the following accession numbers:

### Deposit of Biological Material

| Identification | Accession Number | Date of Deposit |
|---|---|---|
| Pediococcus acidilactici 64-C1 | NRRL Deposit | Apr. 1 2014 |
| | Number B-50962 | |
| Lactobacillus salivarius 64-C2 | NRRL Deposit | Sep. 23 2016 |
| | Number B- 67320 | |
| Lactobacillus salivarius 39B1 | NRRL Deposit | Dec. 22 2015 |
| | Number B-67178 | |
| Pediococcus pentosaceus 39B2 | NRRL Deposit | Dec. 22 2015 |
| | Number B-67179 | |
| Lactobacillus salivarius 59B | NRRL Deposit | Dec. 22 2015 |
| | Number B-67177 | |
| Lactobacillus salivarius 34B | NRRL Deposit | Dec. 22 2015 |
| | Number B-67180 | |

The strains have been deposited under conditions that assure that access to the culture will be available during the pendency of this patent application to one determined by foreign patent laws to be entitled thereto. The deposits represent a substantially pure culture of the deposited strain. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### Description of the Biological Material

Pediococcus acidilactici 64C1 was isolated by Pacific Vet Group from a poultry sample in the US in 2011. The strain was identified as Pediococcus acidilactici based on 16S rDNA sequencing. Lactobacillus salivarius 64-C2 was isolated by Pacific Vet Group from a poultry sample in the US in 2011. The strain was identified as Lactobacillus salivarius based on 16S rDNA sequencing. Lactobacillus salivarius 39B1 was isolated by Pacific Vet Group from a poultry sample in the US in 2011. The strain was identified as Lactobacillus salivarius based on 16S rDNA sequencing. Pediococcus pentosaceus 39B2 was isolated by Pacific Vet Group from a poultry sample in the US in 2011. The strain was identified as Pediococcus pentosaceus based on 16S rDNA sequencing. Lactobacillus salivarius 59B was isolated by Pacific Vet Group from a poultry sample in the US in 2011. The strain was identified as Lactobacillus salivarius based on 16S rDNA sequencing. Lactobacillus salivarius 34B was isolated by Pacific Vet Group from a poultry sample in the US in 2011. The strain was identified as Lactobacillus salivarius based on 16S rDNA sequencing.

### Example 1: Probiotic Bacterial Isolate Screening

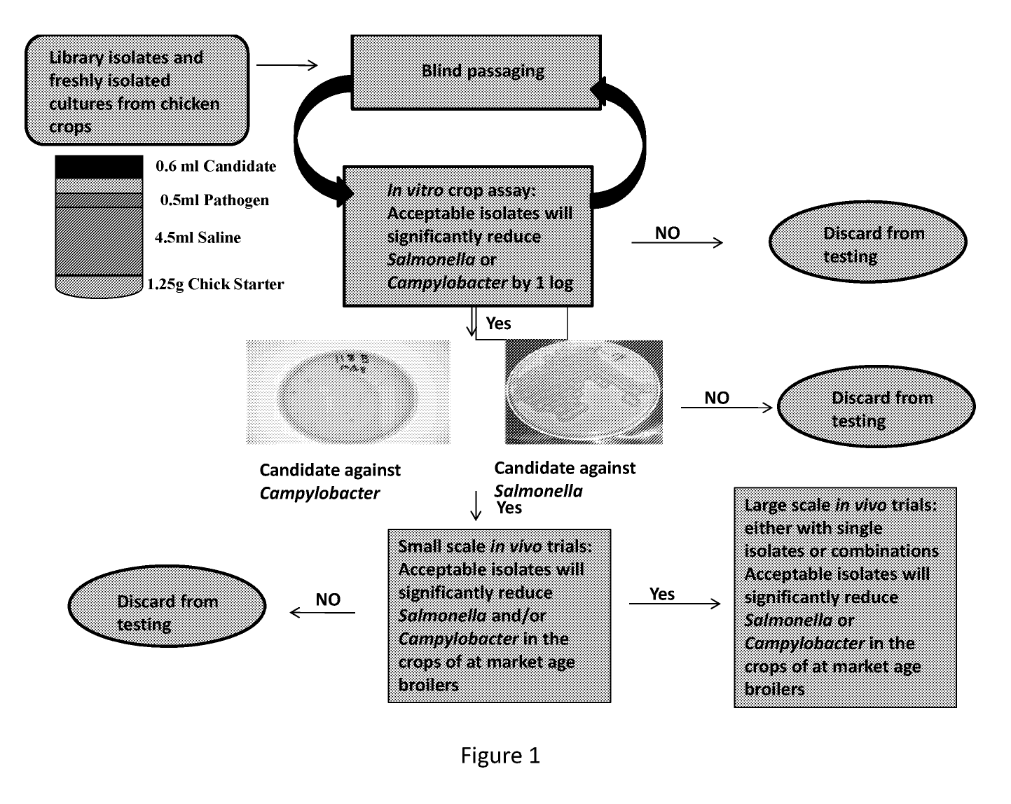

Candidate bacterial isolates were screened as illustrated in FIG. 1. Bacterial isolates were selected from either a library of isolates previously screened for probiotic activity or novel strains isolated from the crop of market age broiler chickens. Bacterial isolates were blind passaged and tested in an in vitro crop assay. Bacterial candidates were tested for the ability to reduce Salmonella or Campylobacter by at least 1 log. Bacterial candidates not possessing this ability were discarded.

In Vitro Crop Assay

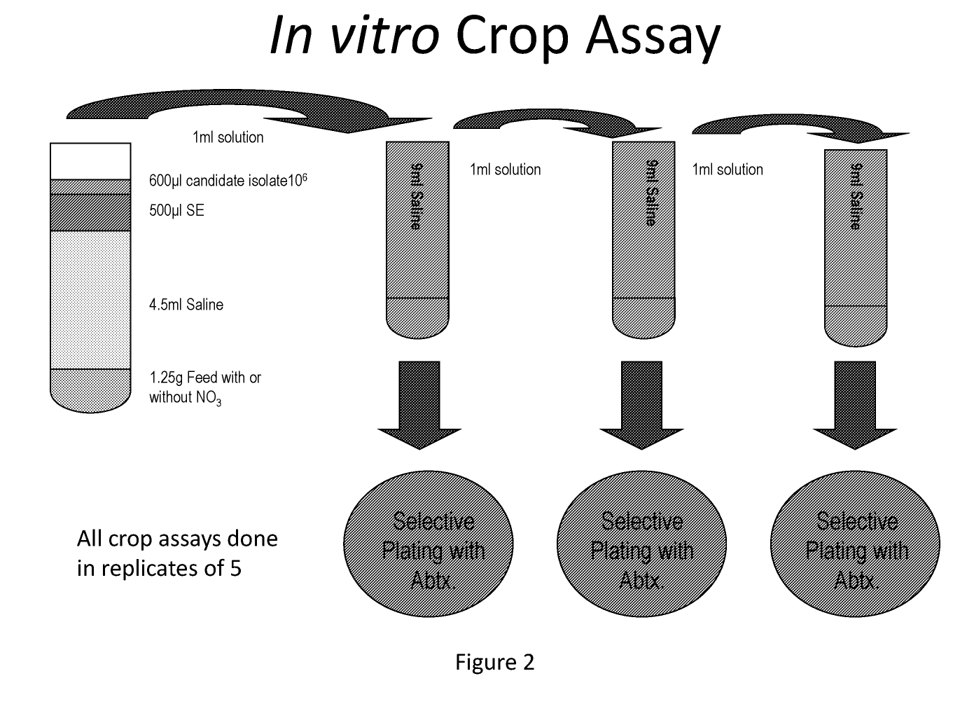

A 600 μl sample of a bacterial candidate (at 1×10⁶) was incubated with 500 μl Salmonella enteritidis (at 10⁵ from an overnight culture) or 500 μl of Campylobacter jejuni (10⁵ from an overnight culture), 4.5 ml saline and 1.25 g of a standard corn-soy based broiler chicken starter feed. The assay was performed in replicates of 5. The samples were serially diluted and plated on agar plates with antibiotics. The bacterial candidates were incubated for 6 hours at 42° C. and cfu/ml (log₁₀) determined (cf. FIG. 2).

### In Vitro Crop Assay Against Salmonella Enteritidis

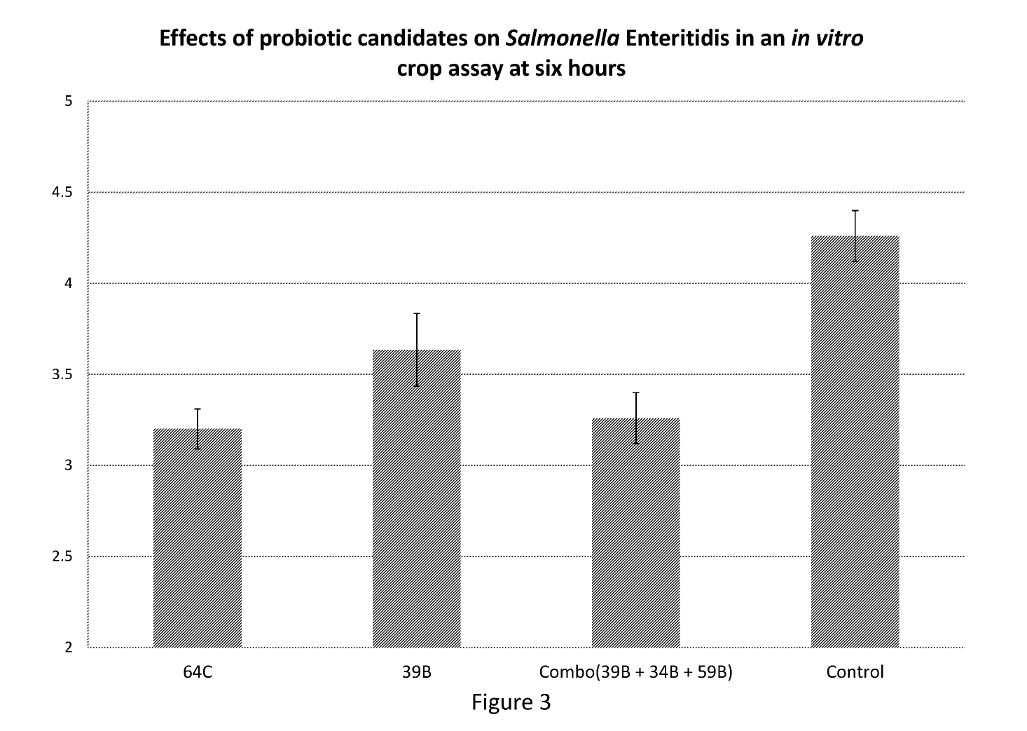

Lactic acid bacteria (LAB) isolates were tested in an in vitro crop assay as described above and in (FIG. 2) and tested against Salmonella enteritidis. LAB isolates and combinations of LAB isolates were incubated with Salmonella enteritidis for 6 hours at 42° C. in the crop assay. After incubation, a sample was taken for enumeration. Samples were serially diluted and plated on brilliant green agar containing novobiocin and nalidixic acid. Plates were incubated at least 18 hours at 37° C. before reading (cf. FIG. 3).

In Vitro Crop Assay Against Campylobacter jejuni

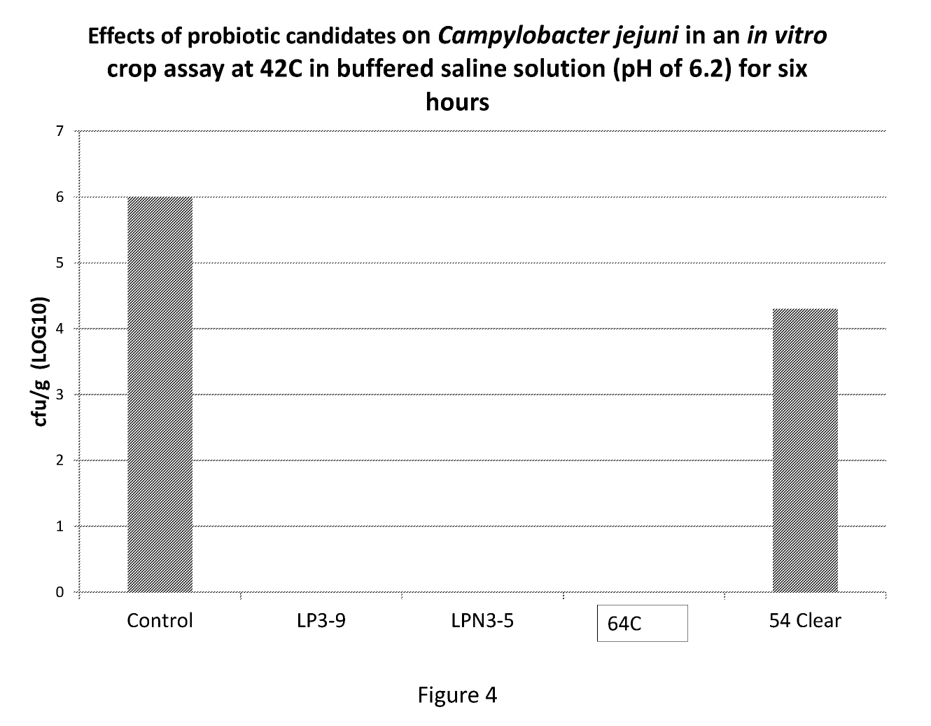

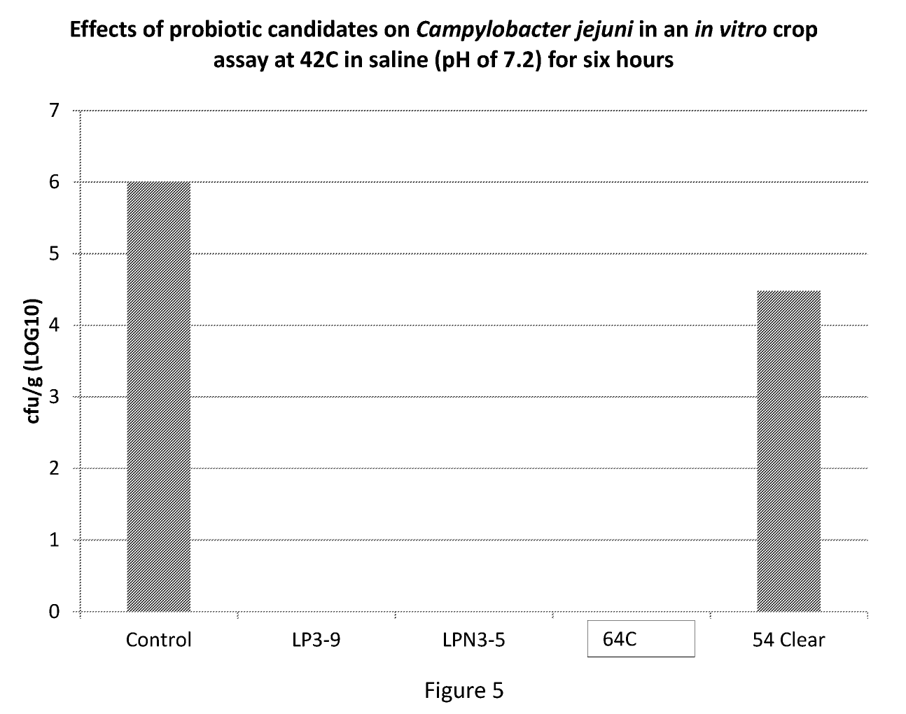

Further lactic acid bacteria (LAB) isolates were selected and tested against Campylobacter jejuni in the crop assay described above and in (FIG. 2). LAB isolates and combinations of LAB isolates were incubated with Campylobacter for 6 hours at 42° C. in buffered saline at pH 6.2 (FIG. 4) or pH 7.2 (saline, FIG. 5) in the crop assay. After incubation, a sample was taken for enumeration. Samples were serially diluted and plated on tryptic soy agar with sheep blood. Plates were incubated at least 18 hours at 37° C. before reading.

### In Vitro Test Against Common Poultry Pathogens.

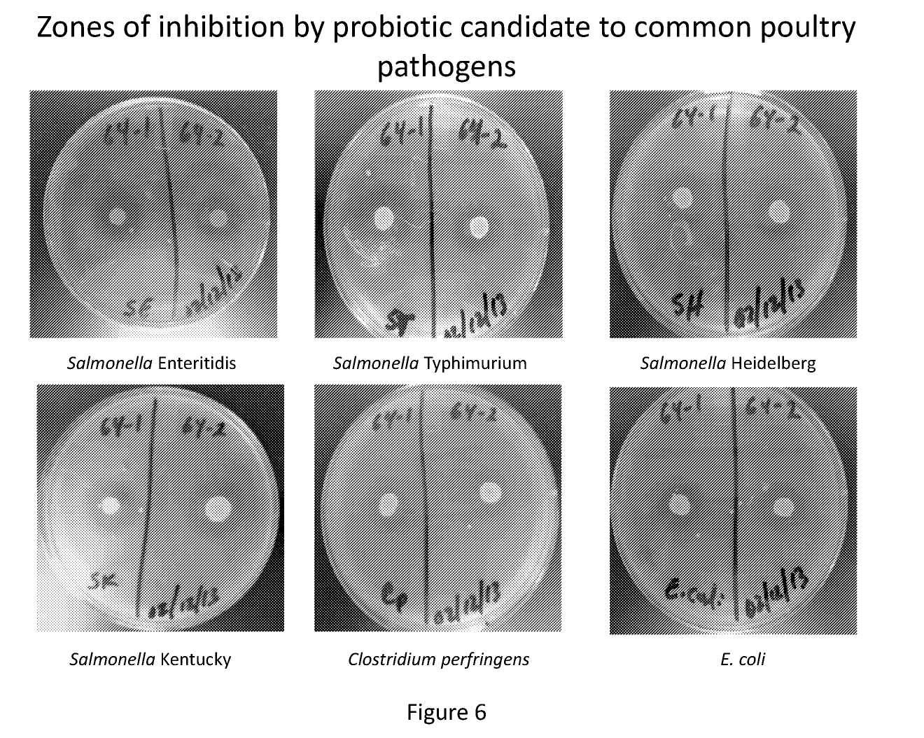

LAB isolates (64-C1 and 64-C2) were incubated on agar plates with Salmonella enteritidis, Salmonella typhimurium, Salmonella heidelberg, Salmonella kentucky, Clostridium perfringens and Escherichia coli and the zone of inhibition determined FIG. 6. Briefly, the LAB isolates were grown in de Man, Rogosa and Sharpe (MRS) broth overnight while being incubated at 37° C. A sterile loop was used to transfer the LAB onto an MRS plate so that a single dense colony will grow on the center of a plate or bi-plate. This plate was incubated for 48 hours at 37° C. During incubation an overnight culture of the pathogen of interest was incubated in appropriate media at 37° C. 100 microliters of the pathogen culture was added to 10 ml of tryptic soy soft agar held at 45° C. The tryptic soy soft agar was poured over the MRS plate containing the LAB isolate and incubated overnight at 37° C.

### Example 2: In Vivo Testing

### Experiment 1

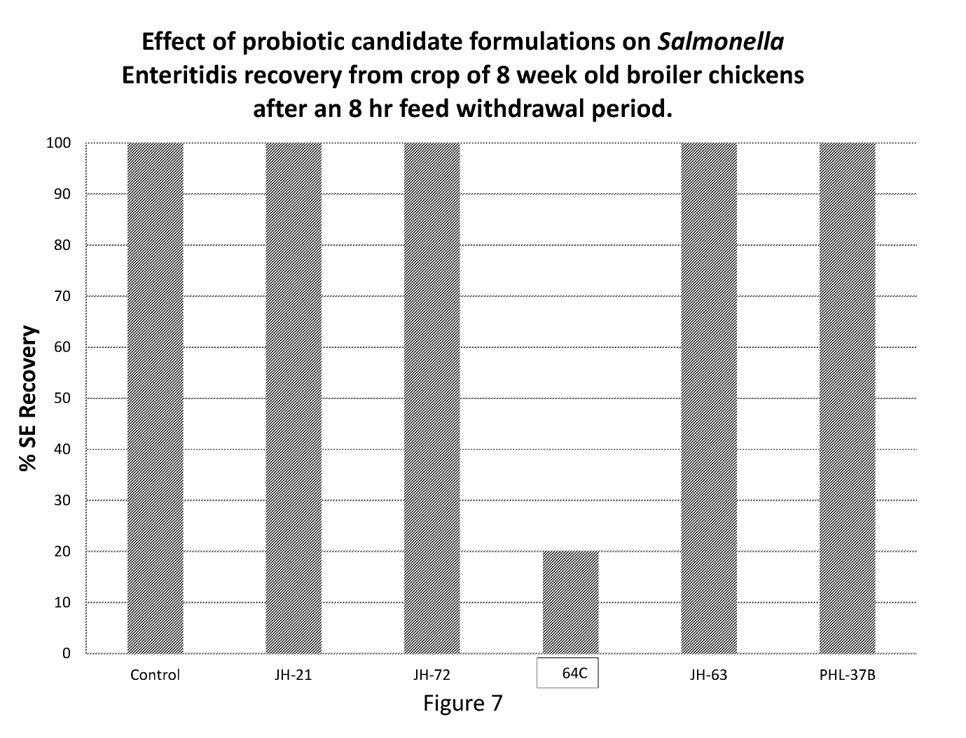

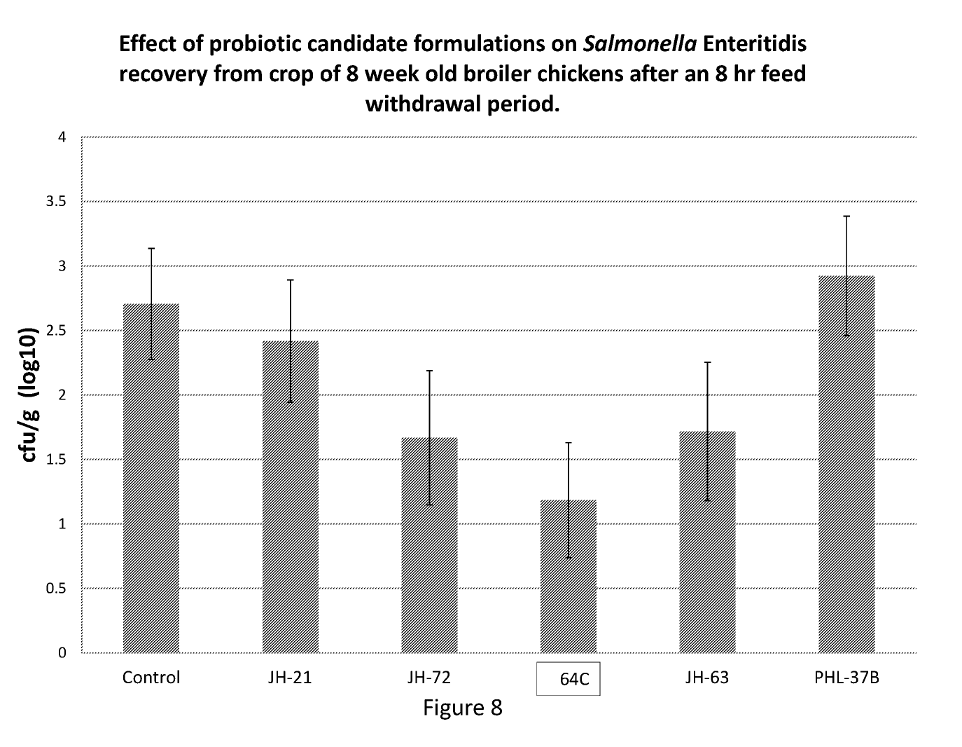

LAB candidates were provided as probiotic formulations to eight week old broiler chickens during an eight (8) hour feed withdrawal period in a small scale single replicate trial (n=12 per group. All LAB formulations were administered via the drinking water at 10⁸ cfu/ml. Chickens were experimentally infected with Salmonella enteritidis the day prior to treatment. The crop of the chickens was removed and tested for the presence of Salmonella enteritidis. Briefly, the chickens were humanely euthanized. The crop was aseptically removed and macerated. Sterile saline was added (4 times the weight of the sample). A sample of the crop/saline solution was taken and serially diluted then directly plated onto brilliant green agar (BGA). To the remainder of the crop/saline solution tetrathionate broth was added to double the initial volume. Both the BGA plates and crop solutions were incubated overnight at 37° C. BGA plates were read and data were recorded. A sample from the crop solution was then struck onto BGA plates and incubated at 37° C. overnight for determination of percent of chickens with Salmonella. FIG. 7 illustrates the percent of chickens from which Salmonella enteritidis was recovered. FIG. 8 illustrates the number of colony forming units (cfu) per gram of cecal content (Log₁₀) of Salmonella enteritidis recovered. In this experiment several LAB formulations (JH-21, JH-72, 64C, JH-63, and PHL-34B were compared to a negative control (Con) that was not treated. In this experiment only on treatment group 64C met the criteria of at least 1 log 10 cfu/g reduction of Salmonella in the crop. Similarly, group 64C was also the only group to reduce incidence as reported as percentage Salmonella positive broiler chickens. This group was selected for further study in multiple replicate trials. These results indicate that not all LAB groups are able to reduce Salmonella within the crop of broiler chickens during a feed withdrawal period.

### Experiment 2

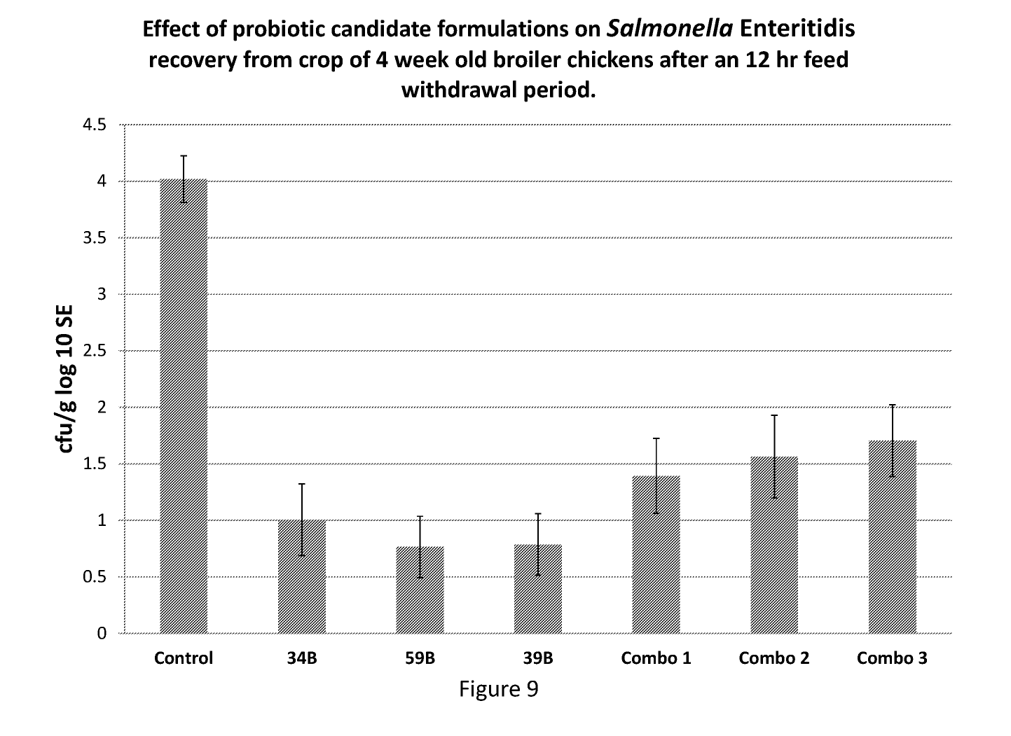

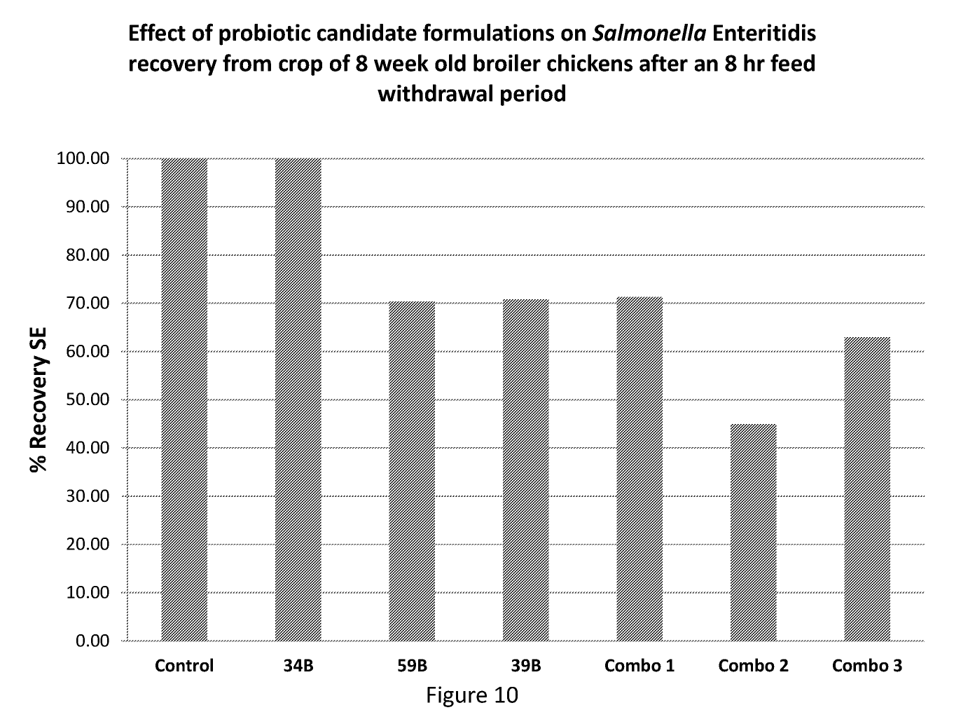

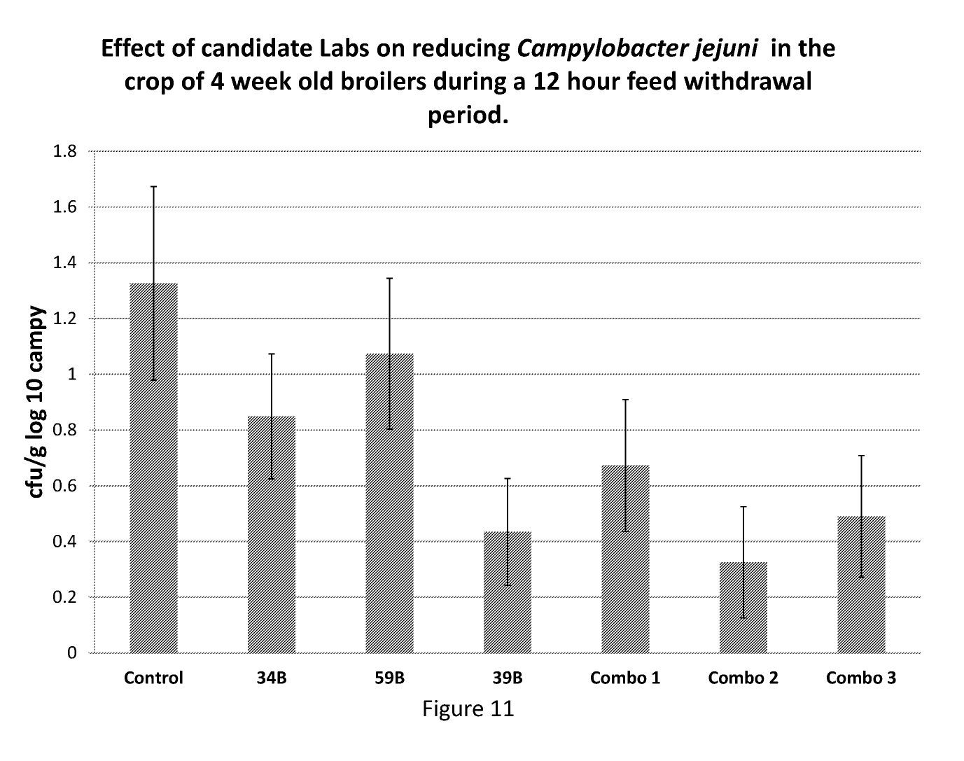

LAB candidates were provided as probiotic formulations to 4 week old broiler chickens for 12 hours (during an eight (8) hour feed withdrawal period and the (4) hours preceding the feed withdrawal. All LAB formulations were administered via the drinking water at 10⁸ cfu/ml. The chickens were infected with Campylobacter jejuni and Salmonella enteritidis prior to treatment. Each bird was gavaged with 10⁸ cfu Salmonella enteritidis the day prior to treatment. Each bird was gavaged with 10⁸ cfu Camylobacter jejuni 7 days prior to treatment. The crops of the chickens were removed and tested for the presence of Salmonella enteritidis and Campylobacter jejuni. Briefly, the chickens were humanely euthanized. The crop was aseptically removed and macerated in a sterile bag. Sterile saline was added (4 times the weight of the sample). A sample of the crop/saline solution was taken and serially diluted then directly plated onto brilliant green agar (BGA) for enumeration of Salmonella or onto Campy-Cephex agar for enumeration of Campylobacter. To the remainder of the crop/saline solution tetrathionate broth was added in an amount to double the initial volume. Both the BGA plates and crops were incubated overnight at 37° C. The Campy-Cephex plates were incubated overnight at 42° C. BGA and Campy-Cephex plates were read and data was recorded. A sample from the crop solution was then struck onto BGA plates and incubated at 37° C. overnight for determination of percent of chickens with Salmonella. FIG. 9 illustrates the number of colony forming units (cfu) per gram (Log₁₀) of Salmonella enteritidis recovered. The crop of the chickens was removed and tested for the presence of Salmonella enteritidis. FIG. 10 illustrates the percent of chickens from which Salmonella enteritidis was recovered. FIG. 11 illustrates the number of colony forming units (cfu) per gram (Log₁₀) of Campylobacter recovered. In this experiment several LAB formulations (34B, 39B, 59B, Combo1, Combo2, and Combo3 were compared to a negative control (Con) that was not treated. In this experiment all treatment groups met the at least 1 log 10 cfu/g reduction of Salmonella in the crop. Similarly, all treatment groups also reduced the incidence as reported as percentage Salmonella positive broiler chickens as compared to the negative control. Groups 39B and Combo2 were selected for further study.

### Multiple Replicate In Vivo Testing Probiotic Formulation

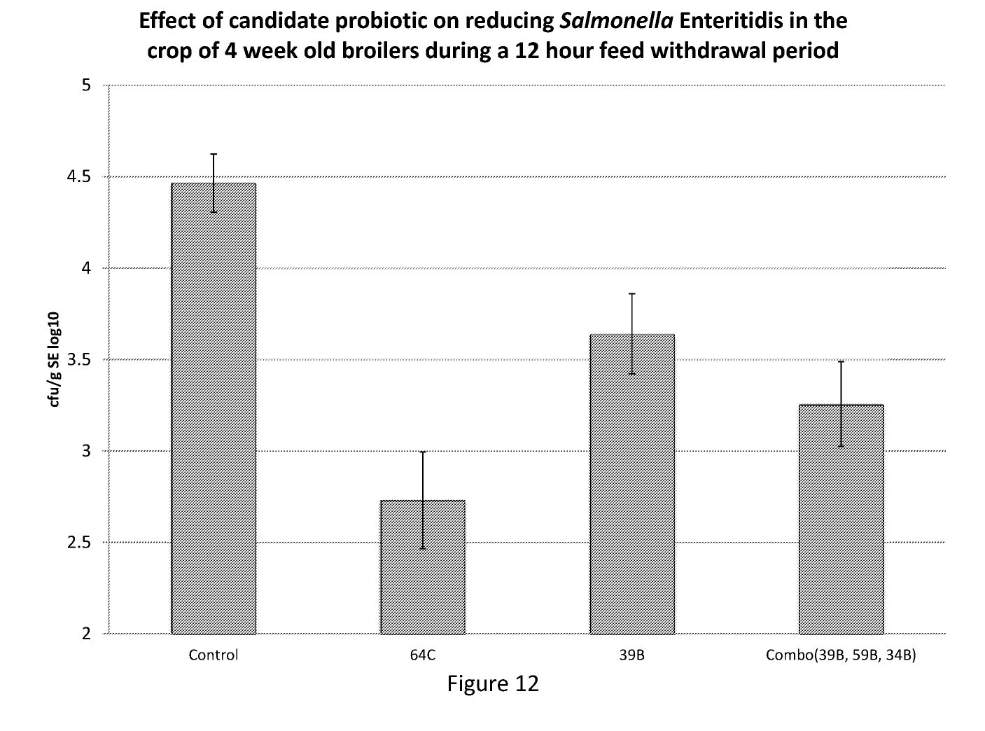

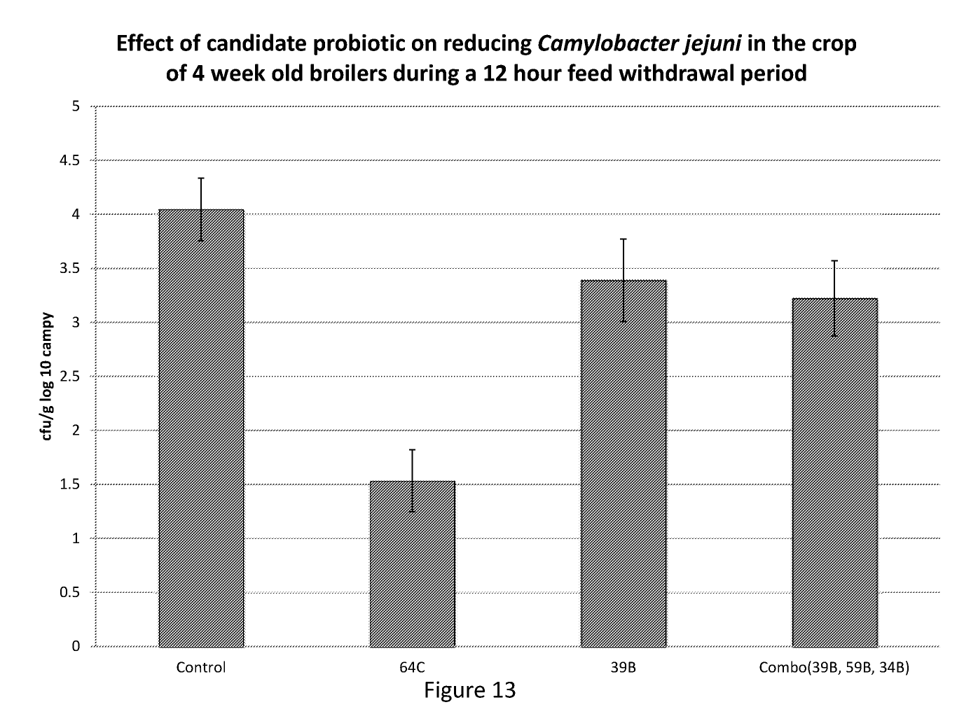

LAB candidates previously tested in single replicate trials that met the minimum 1 log 10 cfu/g reduction of Salmonella from crop content were retested for their ability to reduce Salmonella and Campylobacter in the crop of broiler chickens in a multiple replicate trial. Three replicates pens with 12 chickens each were used for each group. Each bird was gavaged with 10⁸ cfu Salmonella enteritidis the day prior to treatment. Each bird was gavaged with 10⁸ cfu Camylobacter jejuni 7 days prior to treatment. All LAB formulations were administered via the drinking water at 10⁸ cfu/ml. Briefly, the chickens were humanely euthanized. The crop was aseptically removed and macerated. Sterile saline was added (4 times the weight of the sample). A sample of the crop/saline solution was taken and serially diluted then directly plated onto brilliant green agar (BGA) for enumeration of Salmonella or onto Campy-Cephex agar for enumeration of Campylobacter. To the remainder of the crop/saline solution tetrathionate broth was added. Both the BGA plates and crops were incubated overnight at 37° C. The Campy-Cephex plates were incubated overnight at 42° C. BGA and Campy-Cephex cfu plates were read and data were recorded. A sample from the crop solution was then struck onto BGA plates and incubated at 37° C. overnight for determination of percent of chickens with Salmonella. FIG. 12 illustrates the number of colony forming units (cfu) per gram (Log₁₀) of Salmonella enteritidis and FIG. 13 the number of Campylobacter jejuni recovered. In this trial, LAB candidates were compared to a negative control (Con) which was not treated. LAB candidate 64C (Pediococcus acidilactici 64-C1 and Lactobacillus salivarius 64-C2) was prepared as a probiotic formulation using a ratio of 90:10 Pediococcus acidilactici 64-C1: Lactobacillus salivarius 64-C2. Likewise 39B (Pediococcus pentosaceus 39B2 and Lactobacillus salivarius 39B1) was prepared as a probiotic formulation using a ratio of 90:10 for 39B2:39B1. Combo was a combination of Lactobacillus salivarius 59B, Lactobacillus salivarius 34B, and 39B (Pediococcus pentosaceus 39B2 and Lactobacillus salivarius 39B1) in equal proportions. In this study, 64C reduced both Salmonella and Campylobacter more than the other groups. As such, it was selected for use in a commercial field trial in boiler chickens.

### Example 3: Field Trial 1

A total of 16 commercial houses were boot swabbed to identify Salmonella positive houses from two eight-house farms. Houses with more than 6/8 positive Salmonella swabs were selected for inclusion in the trial. Briefly, the houses were split into quadrants and 2 boot swabs were worn while walking the feed and waterlines of a given quadrant. The boots swabs were then removed and placed in a sterile bag and transported on ice back to the laboratory. Swabs were placed into tetrathionate broth and incubated overnight at 37° C. After overnight incubation, a sterile loop was used to streak a sample onto XLT-4 agar which was then incubated overnight at 37° C. Plates were evaluated for presence of Salmonella and data was recorded. Four houses were selected from each farm, two (2) were the controls and two (2) were treated with the probiotic formulation. Each house had approximately 30,000 birds.

TABLE 2
Houses Positive for Salmonella and Treatment

| | Farm 1 | | Farm 2 | |
|---|---|---|---|---|
| | Number Positive | Treatment | Number Positive | Treatment |
| House 1 | 6/8 | ND | 6/8 | Probiotic |
| House 2 | 7/8 | ND | 8/8 | Probiotic |
| House 3 | 8/8 | Probiotic | 3/8 | ND |
| House 4 | 8/8 | Probiotic | 7/8 | Control |
| House 5 | 8/8 | Control | 5/8 | ND |
| House 6 | 8/8 | Control | 8/8 | Control |
| House 7 | 8/8 | ND | 0/8 | ND |
| House 8 | 8/8 | ND | 5/8 | ND |

ND = not determined

Chickens were treated with either control (no probiotic) or with probiotic formulation at the time the feeders were shut off (˜10 hours before live haul). LAB candidate 64C (Pediococcus acidilactici 64-C1 and Lactobacillus salivarius 64-C2) was prepared as a probiotic formulation using a ratio of 90:10 Pediococcus acidilactici 64-C1:Lactobacillus salivarius 64-C2. The probiotic formulation was administered through the waterlines during the feed withdrawal period at 10⁷ cfu/ml.

At live haul, 100 birds were taken from each flock. The birds were transported via cages for approximately 1.5 hours. The crops were removed and cultured for the presence or absence of Salmonella. The crop was aseptically removed and placed into a bag containing tetrationate broth and incubated at 37° C. overnight. A sample from the crop solution was then struck onto XLT-4 plates and incubated at 37° C. overnight for determination of percent of chickens with Salmonella.

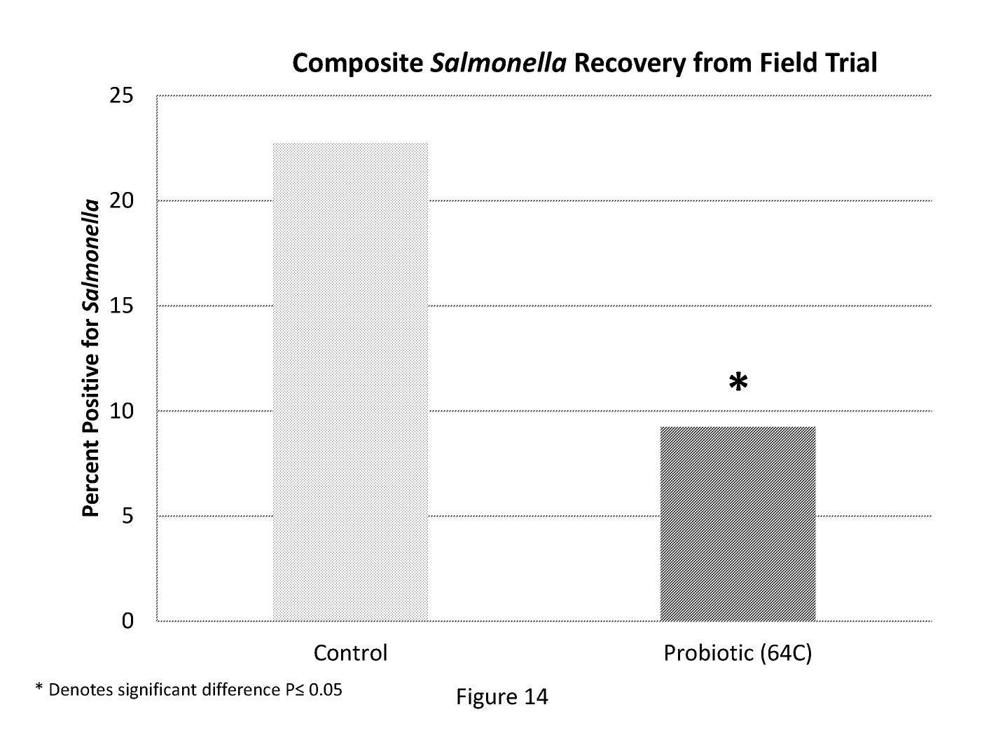

The average of the data demonstrates a significant reduction (P≤0.05) in the number of Salmonella positive birds when treated with the probiotic formulation compared to the control (FIG. 14).

### Example 4: In Vitro Effect on Salmonella Recovery and pH after 24 Hours

### a) Salmonella Enumeration

Enumeration of S. enteritidis was performed at 0 hours, 6 hours, and 24 hours. The Salmonella concentration at 0 hours was similar in all groups. After 6 hours of incubation, no differences in Salmonella recovery was detected between the control groups and LAB 39B2 and LAB 59B. All other LAB's groups were statistically different (P<0.05) compared to the control group. LABS's 64C1, 64C2, and the combination of both isolates highly reduced (P<0.001) Salmonella recovery compared to the other groups after 24 hours of incubation. Also, LAB's 39B1 and 39B2 significantly reduced Salmonella recovery (P<0.05) compared to the control group.

TABLE 2
Effect of different LAB's isolates on Salmonella
recovery in an In Vitro crop assay model.

| | S. enteritidis recovery (Log10) | | |
|---|---|---|---|
| | Mean ± STDERR | | |
| Treatment | 0 h | 6 h | 24 h |
| Control | 2.92 ± 0.05 a | 6.30 ± 0.18 a | 9.23 ± 0.09 a |
| LAB 34B | 2.96 ± 0.09 a | 5.68 ± 0.16 bcd | 8.75 ± 0.07 ab |
| LAB 39B1 | 2.79 ± 0.11 a | 5.69 ± 0.13 bcd | 8.51 ± 0.08 b |
| LAB 39B2 | 2.79 ± 0.10 a | 6.11 ± 0.10 ab | 8.33 ± 0.14 b |
| LAB 59B | 2.89 ± 0.04 a | 5.99 ± 0.07 abc | 8.86 ± 0.08 ab |
| LAB 64C1 | 2.96 ± 0.06 a | 5.36 ± 0.09 d | 6.26 ± 0.24 c |
| LAB 64C2 | 2.85 ± 0.08 a | 5.48 ± 0.15 cd | 6.31 ± 0.12 c |
| LAB 64C1 and | 3.06 ± 0.05 a | 5.37 ± 0.11 d | 6.37 ± 0.19 c |
| LAB 64C2 | | | |
| (50%:50%) | | | |

n = 6 replicates per group

b) pH evaluation

After 24 hours of incubation, pH was recorded with a pH meter (Meter Toledo FiveEasy FE20). All LAB's isolates significantly reduced (p<0.05) the pH of the media compared to the control groups. LAB's 64C1, 64C2, and the combination of both produced a more acidic (P<0.05) environment than LAB's 34B, 39B1, 39B2 and 59B.

TABLE 3
pH evaluation of media with different LAB's isolates
after 24 hours of incubation at 37° C.

| | pH after 24 h incubation |
|---|---|
| Treatment | Mean ± STERR |
| Control | 5.62 ± 0.03 a |
| LAB 34B | 5.41 ± 0.02 b |
| LAB 39B1 | 5.37 ± 0.05 b |
| LAB 39B2 | 5.40 ± 0.06 b |
| LAB 59B | 5.37 ± 0.10 b |
| LAB 64C1 | 3.62 ± 0.01 c |
| LAB 64C2 | 3.64 ± 0.02 c |
| LAB 64C1 and LAB 64C2 (50%:50%) | 3.63 ± 0.02 c |

n = 6 replicates per group

In conclusion, isolates 64C1, 64C2, and the combination of both isolates were very effective to reduced Salmonella recovery after 6 h and 24 h of incubation at 37° C. compared to the control group. These isolates also reduced significantly the pH of the media that may explain their effect on Salmonella recovery. LAB 58B was the only isolate evaluated that did not show any statistical difference at any time point evaluation in this trial although it significantly reduced the pH of media after 24 hours of incubation.

1. A method for reducing the level of Salmonella and/or Campylobacter in poultry meat comprising the step of administering at least one bacterial isolate to the poultry less than 24 hours prior to slaughter, wherein the at least one bacterial isolate is selected to reduce Salmonella and/or Campylobacter in the crop of poultry.

2. The method of claim 1, wherein the at least one bacterial isolate is from the genus Pediococcus, or the genus Lactobacillus, or a combination thereof.

3. The method of claim 1, wherein the at least one bacterial isolate is selected from the group consisting of:
(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;
(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;
(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;
(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;
(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and
(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;
or any combination thereof.

4. The method of claim 1, wherein the Salmonella is Salmonella Enteritidis, Salmonella Typhimurium, Salmonella heidelberg, Salmonella kentucky, or any combination thereof.

5. The method of claim 1, wherein the Campylobacter is Campylobacter jejuni, Campylobacter coli, Campylobacter lari, Campylobacter upsaliensis, or any combination thereof.

6. The method of claim 1, wherein the administration is via the drinking water.

7. The method of claim 1, wherein the administration is performed from 5 to 15 hours prior to slaughter.

8. The method of claim 1, wherein the administration is performed less than 10 hours prior to feed withdrawal.

9. A bacterial isolate for reducing the level of Salmonella and/or Campylobacter in the crop of poultry.

10. The bacterial isolate of claim 9, wherein the bacterial isolate is from the genus Pediococcus or the genus Lactobacillus, or a combination thereof.

11. The bacterial isolate of claim 9, wherein the bacterial isolate is selected from the group consisting of
(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;
(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;
(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;
(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;
(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and
(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;
or any combination thereof.

12. A composition comprising a bacterial isolate of claim 9 and an agriculturally acceptable excipient.

13. The composition of claim 12 comprising at least two bacterial isolates selected from the group consisting of
(a) a Pediococcus acidilactici 64-C1 strain having the deposit accession number NRRL B-50962; a strain having all of the identifying characteristics of Pediococcus acidilactici 64-C1 having the deposit accession number NRRL B-50962 or a mutant thereof;
(b) a Lactobacillus salivarius 64-C2 strain having the deposit accession number NRRL B-67320; a strain having all of the identifying characteristics of Lactobacillus salivarius 64-C2 having the deposit accession number NRRL B-67320 or a mutant thereof;
(c) a Lactobacillus salivarius 39B1 strain having the deposit accession number NRRL B-67178; a strain having all of the identifying characteristics of Lactobacillus salivarius 39B1 having the deposit accession number NRRL B-67178 or a mutant thereof;
(d) a Pediococcus pentosaceus 39B2 strain having the deposit accession number NRRL B-67179; a strain having all of the identifying characteristics of Pediococcus pentosaceus 39B2 having the deposit accession number NRRL B-67179 or a mutant thereof;
(e) a Lactobacillus salivarius 59B strain having the deposit accession number NRRL B-67177; a strain having all of the identifying characteristics of Lactobacillus salivarius 59B having the deposit accession number NRRL B-67177 or a mutant thereof; and
(f) a Lactobacillus salivarius 34B strain having the deposit accession number NRRL B-67180; a strain having all of the identifying characteristics of Lactobacillus salivarius 34B having the deposit accession number NRRL B-67180 or a mutant thereof;
or any combination thereof.

14. (canceled)
